(19) United States
(12) Patent Application Publication
(10) Pub. No.: US 2017/0306417 A1
(43) Pub. Date: Oct. 26, 2017

(54) PREDICTIVE BIOMARKER(S) OF TREATMENT WITH ERB ANTIBODIES

(71) Applicant: Consortium for Clinical Diagnostics, Bethesda, MD (US)

(72) Inventors: Brian LEYLAND-JONES, Montreal (CA); Homer F. WILLIS, Boca Raton, FL (US)

(21) Appl. No.: 15/526,153

(22) PCT Filed: Nov. 12, 2015
(86) PCT No.: PCT/CA2015/051169

§ 371 Date: May 11, 2017

Related U.S. Application Data
(60) Provisional application No. 62/078,487, filed on Nov. 12, 2014.
(60) Provisional application No. 62/088,207, filed on Dec. 5, 2014.

(51) Int. Cl. C12Q 1/68; G01N 33/574; C07K 16/28

(52) U.S. Cl. CPC C12Q 1/6886; G01N 33/57415; G01N 33/57484; C07K 16/2863; C12Q 2600/106; C12Q 2600/118; C12Q 2600/158

(57) ABSTRACT

The present disclosure includes methods for the prediction of outcome in breast cancer where EGFR/ErbB family members are over expressed and the benefit of treatment with an anti-ErbB antibody. More specifically, the present invention relates the use of the mRNA expression level of a C8A, OR56A1, or PRR20C biomarker compared to a reference expression level for providing information regarding the benefit of treatment with a HER2 antibody, such as trastuzumab, in HER2+ breast cancer.

### CROSS-REFERENCE TO RELATED APPLICATIONS

This application claims priority of U.S. provisional patent applications 62/078,487, filed on Nov. 12, 2014, and 62/088,207, filed Dec. 5, 2015, the specifications of which are hereby incorporated by reference in their entireties.

### BACKGROUND

### (a) Field

The present invention relates to a method for the prediction of outcome in breast cancer where EGFR/ErbB family members are over expressed and the benefit of treatment with an anti-ErbB antibody. More specifically, the present invention relates the use of the mRNA expression level of a C8A, OR56A1, or PRR20C biomarker compared to a reference expression level for providing information regarding the benefit of treatment with a HER2 antibody, such as trastuzumab, in HER2+ breast cancer.

### (b) Related Prior Art

Biomarker can help diagnose various tumors and sometimes determine the response to therapy or recurrence. An ideal biomarker would be specific for a given tumor type, be detectable at low levels in the tumor cell, have a direct relationship to the type of disease, and be present in all subjects with the tumor. However, no biomarker has all the requisite characteristics to provide enough specificity or sensitivity to be used in early diagnosis or mass cancer screening programs.

Oncologists have a number of treatment options available to them, including different combinations of chemotherapeutic drugs that are characterized as “standard of care,” and a number of drugs that do not carry a label claim for particular cancer, but for which there is evidence of efficacy in that cancer. Best likelihood of good treatment outcome requires that patients be assigned to optimal available cancer treatment, and that this assignment be made as quickly as possible following diagnosis.

mRNA-based tests have not often been used because of the problem of mRNA degradation over time and the fact that it is difficult to obtain fresh tissue samples from patients for analysis. Fixed paraffin-embedded tissue is more readily available and methods have been established to detect RNA in fixed tissue. However, these methods typically do not allow for the study of large numbers of transcribed genes (mRNA) from small amounts of material. Thus, traditionally fixed tissue has been rarely used other than for immunohistochemistry detection of proteins.

Certain classifications of human breast cancers based on gene expression patterns have also been reported. However, these studies mostly focus on improving and refining the already established classification of various types of cancer, including breast cancer, and generally do not provide new insights into the relationships of the differentially expressed genes, and do not link the findings to treatment strategies in order to improve the clinical outcome of cancer therapy.

Although modern molecular biology and biochemistry have revealed hundreds of genes whose activities influence the behavior of tumor cells, state of their differentiation, and their sensitivity or resistance to certain therapeutic drugs, with a few exceptions, the status of these genes has not often been exploited for the purpose of routinely making clinical decisions about drug treatments. One notable exception is the use of estrogen receptor (ER) protein expression in breast carcinomas to select patients to treatment with anti-estrogen drugs, such as tamoxifen.

Despite recent advances, the challenge of cancer treatment remains to target specific treatment regimens to pathogenically distinct tumor types, and ultimately personalize tumor treatment in order to maximize outcome. Hence, a need exists for tests that simultaneously provide predictive information about patient responses to the variety of treatment options. This is particularly true for breast cancer, the biology of which is poorly understood. It is clear that the classification of breast cancer into a few subgroups, such as ERBB2+/HeR2+ subgroup, and subgroups characterized by low to absent gene expression of the estrogen receptor (ER) and a few additional transcriptional factors (Perou et al., Nature 406:747-752 (2000)) does not reflect the cellular and molecular heterogeneity of breast cancer, and does not allow the design of treatment strategies maximizing patient response. Breast cancer is the second most common cause of cancer-related mortality in Western women. One of the important challenges in current breast cancer research is to develop effective methods to determine whether a patient is likely to have a recurrence or progress to the aggressive, metastatic disease in order to aid clinicians in deciding the appropriate course of treatment. This is especially true for women with so called triple negative (TN) breast cancer. These tumors can be identified by the fact that they do not express the estrogen or progesterone receptors and express only normal (not amplified) levels of the human epidermal growth factor receptor 2 (HER2). However, it is currently impossible to predict the outcome of TN patients based solely on the pathological evaluation of the tumor.

Members of the ErbB family of receptor tyrosine kinases are important mediators of cell growth, differentiation and survival. The receptor family includes four distinct members, including epidermal growth factor receptor (EGFR or ErbBD, HER2 (ErbB2 or p185^{m}″), HER3 (ErbB3) and HER4 (ErbB4 or tyro2). p185, was originally identified as the product of the transforming gene from neuroblastomas of chemically treated rats. The activated form of the neu proto-oncogene results from a point mutation (valine to glutamic acid) in the transmembrane region of the encoded protein. Amplification of the human homolog of neu is observed in breast and ovarian cancers and correlates with a poor prognosis (Slamon et al., Science, 235:177-182 (1987); Siamon et al., Science, 244:707-712 (1989); and U.S. Pat. No. 4,968,603). To date, no point mutation analogous to that in the /rø/ proto-oncogene has been reported for human tumors. Overexpression of ErbB2 (frequently but not uniformly due to gene amplification) has also been observed in other carcinomas including carcinomas of the stomach, endometrium, salivary gland, lung, kidney, colon, thyroid, pancreas and bladder. See, among others, King et al., Science, 229:974 (1985); Yokota et al., Lancet: 1:765-767 (1986); Fukushigi et al., Mol Cell Biol., 6:955-958 (1986); Geurin et al., Oncogene Res., 3:21-31 (1988); Cohen et al., Oncogene, 4:81-88 (1989); Yonemura et al., Cancer Res., 51:1034 (1991); Borst et al., Gynecol. Oncol., 38:364 (1990); Weiner et al., Cancer Res., 50:421-425 (1990); Kern et al., Cancer Res., 50:5184 (1990); Park et al., Cancer Res., 49:6605 (1989); Zhau et al, Mol. Carcinog., 3:354-357 (1990); Aasland et al. Br. J. Cancer 57:358-363 (1988); Williams et al. Pathobiology 59:46-52 (1991); and McCann et al., Cancer, 65:88-92 (1990). ErbB2 may be overexpressed in prostate cancer (Gu et al. Cancer Lett. 99:185-9 (1996); Ross et al. Hum. Pathol. 28:827-33 (1997); Ross et al. Cancer 79:2162-70 (1997); and Sadasivan et al. J. Urol. 150:126-31 (1993)). A spliced form of erbB2 oncogene encoding a constitutively tyrosine phosphorylated ErbB2 receptor is disclosed in PCT publication WO00/20579, published on Apr. 13, 2000. The erbB2 protein encoded by the splice variant has an in frame deletion of 16 amino acids (CVDLDDKGCPAEQRAS), two of which are conserved cysteine residues.

A variety of anti-ErbB antibody are known in the prior art, and a variety of these antibodies are anti-HER2 antibodies. Such antibodies are preferably monoclonal antibodies. They may either be so-called chimeric antibodies, humanized antibodies or fully human antibodies. They may either be full length anti-HER2 antibodies; anti-HER2 antibody fragments having the same biological activity; including amino acid sequence variants and/or glycosylation variants of such antibodies or fragments. Examples of humanized anti-HER2 antibodies are known under the INN names Trastuzumab and Pertuzumab. Another suitable anti-HER2 antibody is T-DM1, which is an antibody-toxin conjugate consisting of huMAb4D5-8 (HERCEPTIN™) and a maytansinoide (viz. DM1=N2′-deacetyl-N2′-(3-mercapto-1-oxopropyl)-maytansine; a highly potent antimicrotubule agent) which conjugate (with a MCC linker) is currently under development for metastatic breast cancer. Other HER2 antibodies with various properties have been described in Tagliabue et al., Int. J. Cancer, 47:933-937 (1991); McKenzie et al., Oncogene, 4:543-548 (1989); Cancer Res., 51:5361-5369 (1991); Bacus et al., Molecular Carcinogenesis, 3:350-362 (1990); Stancovski et al., PNAS (USA), 88:8691-8695 (1991); Bacus et al., Cancer Research, 52:2580-2589 (1992); Xu et al., Int. J. Cancer, 53:401-408 (1993); WO94/00136; Kasprzyk et al., Cancer Research, 52:2771-2776 (1992); Hancock et al., Cancer Res., 51:4575-4580 (1991); Shawver et al., Cancer Res., 54:1367-1373 (1994); Arteaga et al., Cancer Res., 54:3758-3765 (1994); Harwerth et al., J. Biol. Chem., 267:15160-15167 (1992); U.S. Pat. No. 5,783,186; and Klapper et al., Oncogene, 14:2099-2109 (1997). The most successful therapeutic anti-HER2 antibody is Trastuzumab sold by Genentech Inc. and F. Hoffmann-La Roche Ltd under the trade name HERCEPTIN™. Further details on the HER2 antigen and antibodies directed thereto are described in many patent and non-patent publications (for a suitable overview see U.S. Pat. No. 5,821,337 and WO 2006/044908).

Accordingly, novel methods of determining or classifying breast cancer subtypes are highly desirable.

Also, novel methods of predicting the benefit of treatment with a drug on breast cancer are highly desirable.

### SUMMARY

According to an embodiment, there is provided a method for prognosis of breast cancer outcome and/or predicting the benefit of treatment with an anti-ErbB antibody, comprising the step of:
(a) providing information on breast cancer treatment based on a comparison of mRNA expression level of a gene signature consisting of at least one biomarker chosen from C8A, OR56A1, PRR20C, B3GNT7, MIR609, ADAMTS7, MIR188, DSG3, FLJ45139, FRMPD2, MIR1913, MIR631, PNLIPRP1, TAT, ZNF528, OR52W1, LOC100131187, PTGS1, GFI1B, and TLX2 in a test sample from a subject, to a reference expression level of the biomarker,
   wherein the biomarker expression level may be quantified using a biological assay;
   wherein over expression of the identified biomarker in the test sample indicates a poor breast cancer outcome and a beneficial treatment with the anti-ErbB antibody, and
   wherein minimal expression, or under expression of the biomarker in the test sample indicates positive outcome and a lack of benefit of treatment with the anti-ErbB antibody.

The method may further comprises step (b) when the gene signature comprises at least two biomarkers:
(b) optimizing the gene signature by identifying a common biological attribute between the at least two biomarker, to provide an optimized gene signature,
   wherein over expression in the test sample of the at least two biomarkers identified in the optimized gene signature indicates a poor breast cancer outcome and a beneficial treatment with ErbB antibodies, and
   wherein minimal expression, or under expression in the test sample of the at least two biomarkers identified in the optimized gene signature indicates positive outcome and a lack of benefit of treatment with ErbB antibodies.

The biomarker may be one of C8A, OR56A1, PRR20C, or a combination of C8A, OR56A1 and PRR20C.

The biomarker may be C8A.

According to another embodiment, there is provided a method for treatment of cancer in an individual comprising:
a) determining, in a tumor sample from the patient, a value for at least one biomarker chosen from C8A, OR56A1, PRR20C, B3GNT7, MIR609, ADAMTS7, MIR188, DSG3, FLJ45139, FRMPD2, MIR1913, MIR631, PNLIPRP1, TAT, ZNF528, OR52W1, LOC100131187, PTGS1, GFI1B, and TLX2;
b) comparing the value obtained in step a) for the biological marker with a predetermined reference value for the same biomarker; and
c) administering a therapeutic agent to the individual if the value of the biomarker is above/below the predetermined reference value.

According to another embodiment, there is provided a method of identifying a cancer patient suitable for treatment with a therapeutic agent, comprising:
a) determining, in a tumor sample from the patient, a value for at least one biomarker chosen from C8A, OR56A1, PRR20C, B3GNT7, MIR609, ADAMTS7, MIR188, DSG3, FLJ45139, FRMPD2, MIR1913, MIR631, PNLIPRP1, TAT, ZNF528, OR52W1, LOC100131187, PTGS1, GFI1B, and TLX2;
b) comparing the value obtained in step a) for the biological marker with a predetermined reference value for the same biomarker; and
c) identifying the cancer patient as suitable for treatment with the therapeutic agent if the value of the biomarker is above/below the predetermined reference value.

According to another embodiment, there is provided a method for the prognosis of cancer, comprising:
a) determining, in a tumor sample from the patient, a value for at least one biomarker chosen from C8A, OR56A1, PRR20C, B3GNT7, MIR609, ADAMTS7, MIR188, DSG3, FLJ45139, FRMPD2, MIR1913, MIR631, PNLIPRP1, TAT, ZNF528, OR52W1, LOC100131187, PTGS1, GFI1B, and TLX2; and
b) comparing the value obtained in step a) for the biomarker with a predetermined reference value for the same biological marker, which predetermined reference value is correlated with a specific prognosis of cancer;
wherein when the value of the biomarker is above the predetermined reference value, prognosis of the cancer outcome is a bad prognosis; and wherein when the value of the biomarker is below the predetermined reference value, prognosis of the cancer outcome is a good prognosis.

The method may be an in vitro method.

The method may further comprise step (b) when the gene signature comprises at least two biomarkers:
(b) optimizing the gene signature by identifying a common biological attribute between the at least two biomarker, to provide an optimized gene signature,
   wherein over expression in the test sample of the at least two biomarkers identified in the optimized gene signature indicates a poor breast cancer outcome and a beneficial treatment with ErbB antibodies, and
   wherein minimal expression, or under expression in the test sample of the at least two biomarkers identified in the optimized gene signature indicates positive outcome and a lack of benefit of treatment with ErbB antibodies.

The biomarker may be one of C8A, OR56A1, or PRR20C, or a combination of C8A, OR56A1 and PRR20C.

The biomarker may be C8A.

The biomarker is a combination of FRMPD2, PTGS1, OR52W1, MIR631, and DSG3, or a combination of TAT, MIR1913, PNLIPRP1, ADAMTS7, and MIR188, or a combination of OR52W1, B3GNT7, and OR56A1.

The cancer may be breast cancer.

The method of the present invention, wherein when the value of the biomarker is above the predetermined reference value, the therapeutic agent may be an anti-ErbB antibody.

The method of the present invention, wherein when the value of the biomarker is below the predetermined reference value, the therapeutic agent may be different than an anti-ErbB antibody.

The method may further comprise treatment with an additional therapeutic agent.

The therapeutic agent different than an anti-ErbB antibody or the additional therapeutic agent may be at least one of doxorubicin (Adriamycin®), Liposomal doxorubicin (Doxil®), epirubicin (Ellence®), taxanes such as paclitaxel (Taxol®), docetaxel (Taxotere®), Albumin-bound paclitaxel (nab-paclitaxel or Abraxane®), fluorouracil (5-FU), cyclophosphamide (Cytoxan®), platinum agents such as cisplatin and carboplatin, Vinorelbine (Navelbine®), Capecitabine (Xeloda®), Gemcitabine (Gemzar®), Mitoxantrone; Ixabepilone (Ixempra®), Eribulin (Halaven®).

The value for a biological marker may be an expression level.

The determining may be with a biological assay.

The biological assay may be at least one of PCR based methods, hybridization based methods, sequencing methods, protein detection methods, or combinations thereof.

The PCR based methods comprise reverse transcriptase polymerase chain reaction (RT-PCR), and quantitative reverse transcriptase polymerase chain reaction (QRT-PCR).

The hybridization methods comprise nuclease protection assay, Northern blot analysis, in situ hybridization, and microarray based analysis.

The sequencing methods comprises next generation sequencing (NGS) technologies.

The protein detecting methods comprises a Western blot analysis, an enzyme-linked immunosorbent assay (ELISA), immunohistochemistry analysis, an immunoprecipitation followed by an SDS-PAGE analysis, a proteomic analysis.

The expression level may be one of an mRNA expression level, a protein expression level, or combinations thereof.

The value for a biological marker may be a gene copy number.

The value of the biomarker is above the predetermined reference value, the bad prognosis may be indicative of a beneficial treatment with an anti-ErbB antibody.

The value of the biomarker is below the predetermined reference value the good prognosis may be indicative of a non-beneficial treatment with an anti-ErbB antibody.

The anti-ErbB antibody may be one or more than one anti-ErbB antibody.

The one or more anti-ErbB antibody may be Pertuzumab, Trastuzumab, T-DM1 or combinations thereof.

The reference value for the at least one biomarker or the reference expression level of the biomarker may be determined by a method comprising the steps:
a) providing at least one collection of tumor samples selected from the group consisting of:
   i) a collection of tumor samples from cancer patients with a known cancer classification, having undergone anti-cancer treatment, and subsequently having no cancer relapse or no cancer recurrence after the anti-cancer treatment;
   ii) a collection of tumor samples from cancer patients with a known cancer classification, having undergone anti-cancer treatment, and subsequently having cancer relapses or recurrences after the anti-cancer treatment.
b) quantifying, for each sample comprised in a collection of tumor samples provided at step a), the at least one biomarker, whereby a collection of quantification values for the at least one biomarker and for the collection of tumor samples is obtained;
c) calculating, from the collection of quantification values obtained at the end of step b), the mean quantification value for the at least one biomarker, whereby a predetermined reference value for the at least one biomarker that is correlated with a specific cancer prognosis is obtained.

The reference value for the at least one biomarker or the reference expression level of the biomarker is a reference cut-off value determined by a method comprising the steps:
a) selecting at least one biomarker for which a reference value is to be determined;
b) providing a collection of tumor tissue samples from cancer patients;
c) providing, for each tumor sample provided at step b), information relating to the actual clinical outcome for the corresponding cancer patient;
d) providing a serial of arbitrary quantification values for the at least one biomarker selected at step a);
e) quantifying the at least one biomarker in each tumor tissue sample contained in the collection provided at step b);
f) classifying the tumor samples in two groups for one specific arbitrary quantification value provided at step c), respectively:
   (i) a first group comprising tumor samples that exhibit a quantification value for the at least one biomarker that is lower than the arbitrary quantification value contained in the serial of quantification values;
   (ii) a second group comprising tumor samples that exhibit a quantification value for the at least one biomarker that is higher than the arbitrary quantification value contained in the serial of quantification values;
   whereby two groups of tumor samples are obtained for the specific quantification value, wherein the tumors samples of each group are separately enumerated;
g) calculating the statistical significance between (i) the quantification value for the at least one biomarker obtained at step e) and (ii) the actual clinical outcome of the patients from which tumor samples contained in the first and second groups defined at step f) derive;
h) reiterating steps f) and g) until every arbitrary quantification value provided at step d) is tested;
i) setting the reference cut off value as consisting of the arbitrary quantification value for which the highest statistical significance (most significant) has been calculated at step g).

In the method of the present invention, in step c), the information relating to the actual clinical outcome of the patients is selected from the group consisting of the duration of the disease-free survival (DFS) and the overall survival (OS), and a combination thereof.

The reference cut-off value is a median quantification value for the at least one biomarker that discriminates between bad cancer prognosis and good cancer prognosis.

The method of the present invention, wherein when the at least one biomarker is an expression level of a gene, the reference value of the gene expression value or the reference expression level of the biomarker correlates with bad cancer prognosis.

According to another embodiment, there is provided a kit for the prognosis of breast cancer outcome and/or predicting the benefit of treatment with an anti-ErbB antibody, comprising
(a) an agent that specifically detects a biomarker, wherein the biomarker is at least one biomarker chosen from C8A, OR56A1, PRR20C, B3GNT7, MIR609, ADAMTS7, MIR188, DSG3, FLJ45139, FRMPD2, MIR1913, MIR631, PNLIPRP1, TAT, ZNF528, OR52W1, LOC100131187, PTGS1, GFI1B, and TLX2; and
(b) instructions to use the kit.

According to another embodiment, there is provided a kit for identifying a cancer patient suitable for treatment with a therapeutic agent comprising:
(a) an agent that specifically detects a biomarker, wherein the biomarker may be at least one biomarker chosen from C8A, OR56A1, PRR20C, B3GNT7, MIR609, ADAMTS7, MIR188, DSG3, FLJ45139, FRMPD2, MIR1913, MIR631, PNLIPRP1, TAT, ZNF528, OR52W1, LOC100131187, PTGS1, GFI1B, and TLX2; and
(b) instructions to use the kit.

According to another embodiment, there may be provided a kit for the prognosis of cancer comprising:
(a) an agent that specifically detects a biomarker, wherein the biomarker may be at least one biomarker chosen from C8A, OR56A1, PRR20C, B3GNT7, MIR609, ADAMTS7, MIR188, DSG3, FLJ45139, FRMPD2, MIR1913, MIR631, PNLIPRP1, TAT, ZNF528, OR52W1, LOC100131187, PTGS1, GFI1B, and TLX2; and
(b) instructions to use the kit.

The agent that specifically detects a biomarker may be a nucleic acid probe, a protein binding agent, or a combination thereof.

The nucleic acid probe may be a pair of nucleic acid probe.

The protein binding agent may be an antibody, an antibody fragment, an aptamer, or a combination thereof.

The biomarker may be C8A.

Features and advantages of the subject matter hereof will become more apparent in light of the following detailed description of selected embodiments, as illustrated in the accompanying figures. As will be realized, the subject matter disclosed and claimed is capable of modifications in various respects, all without departing from the scope of the claims. Accordingly, the drawings and the description are to be regarded as illustrative in nature, and not as restrictive and the full scope of the subject matter is set forth in the claims.

### BRIEF DESCRIPTION OF THE DRAWINGS

Further features and advantages of the present disclosure will become apparent from the following detailed description, taken in combination with the appended drawings, in which:

FIG. 1 shows a Kaplan Meier Survival Analysis for the TransHERA Full Cohort [C8A]-Sum<=595.3, comparing observations vs treatment.

FIG. 2 shows a Kaplan Meier Survival Analysis for the TransHERA Full Cohort [C8A]-Sum>595.3, comparing observations vs treatment.

FIG. 3 shows a Kaplan Meier Survival Analysis for the TransHERA Full Cohort [C8A], treatment arm.

FIG. 4 shows a Kaplan Meier Survival Analysis for the TransHERA Full Cohort [C8A], observation arm.

FIG. 5 shows a Kaplan Meier Survival Analysis for the TransHERA C8A Low Observation vs High Treatment.

FIG. 6 shows C8A Expression TransHERA Cohort.

FIG. 7 shows a Kaplan Meier Survival Analysis for the DFS [C8A, C9]-Observation.

FIG. 8 shows a Kaplan Meier Survival Analysis for the DFS [C8A, C9] treatment year 1 & 2.

FIG. 9 shows a Kaplan Meier Survival Analysis for the DFS [C8A, C9] High Expression.

FIG. 10 shows a Kaplan Meier Survival Analysis for the DFS [C8A, C9] Low Expression.

FIG. 11 shows Observation [C8A, C9].

FIG. 12 shows the Biocarta Lectin Pathway.

FIG. 13 shows the (A) C8A/C9 low expression group and (B) C8A/C9 high expression group.

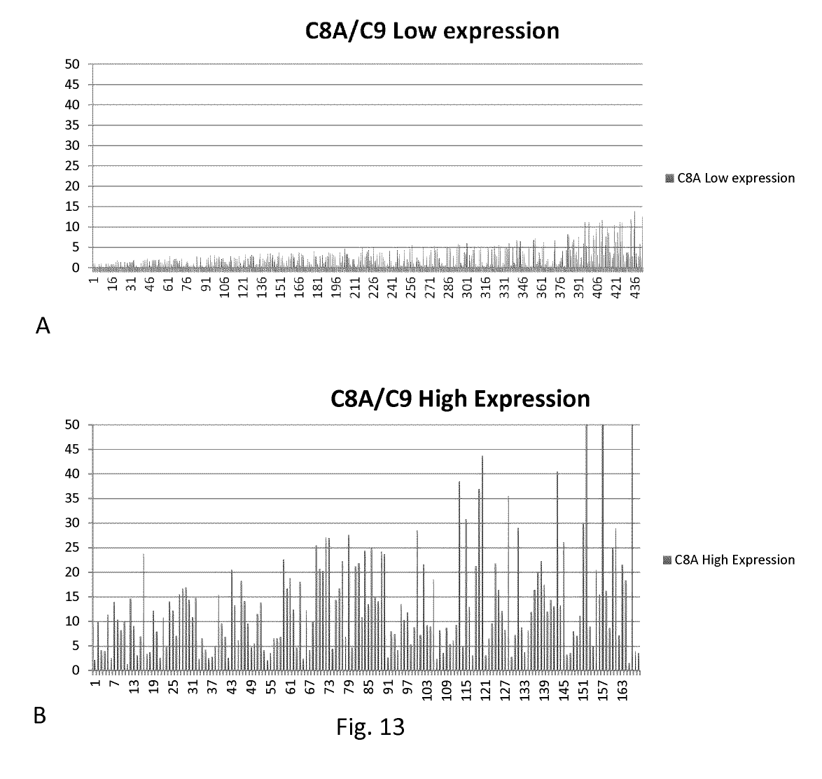

FIG. 14 shows the Percent Sample Alteration for Each Cancer Study with Mutation Data (C8A).

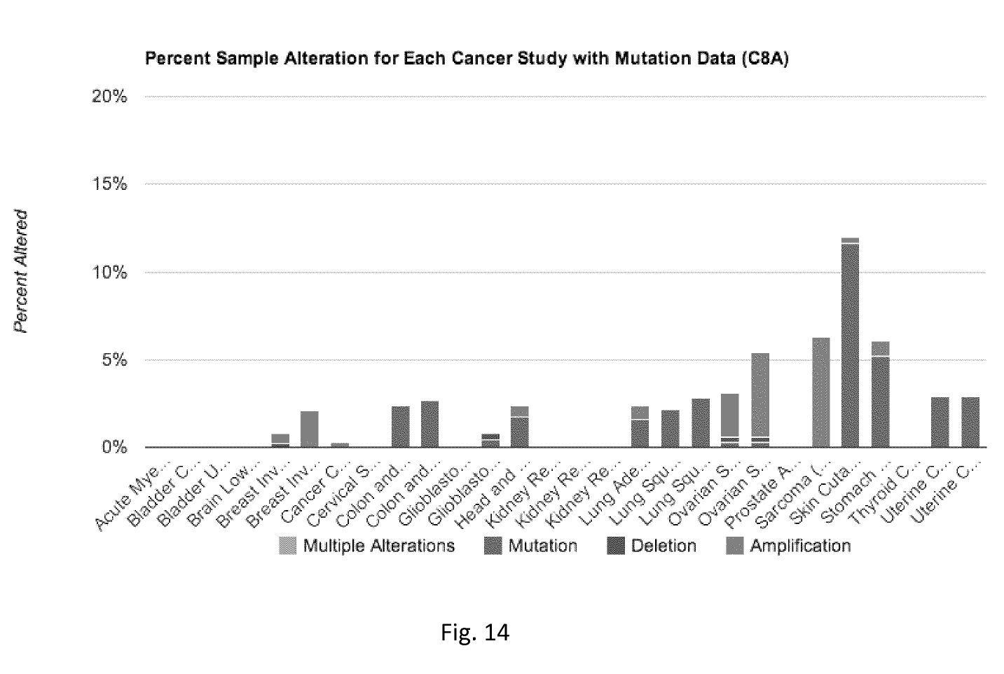

FIG. 15 shows the C8A expression across different tissue types.

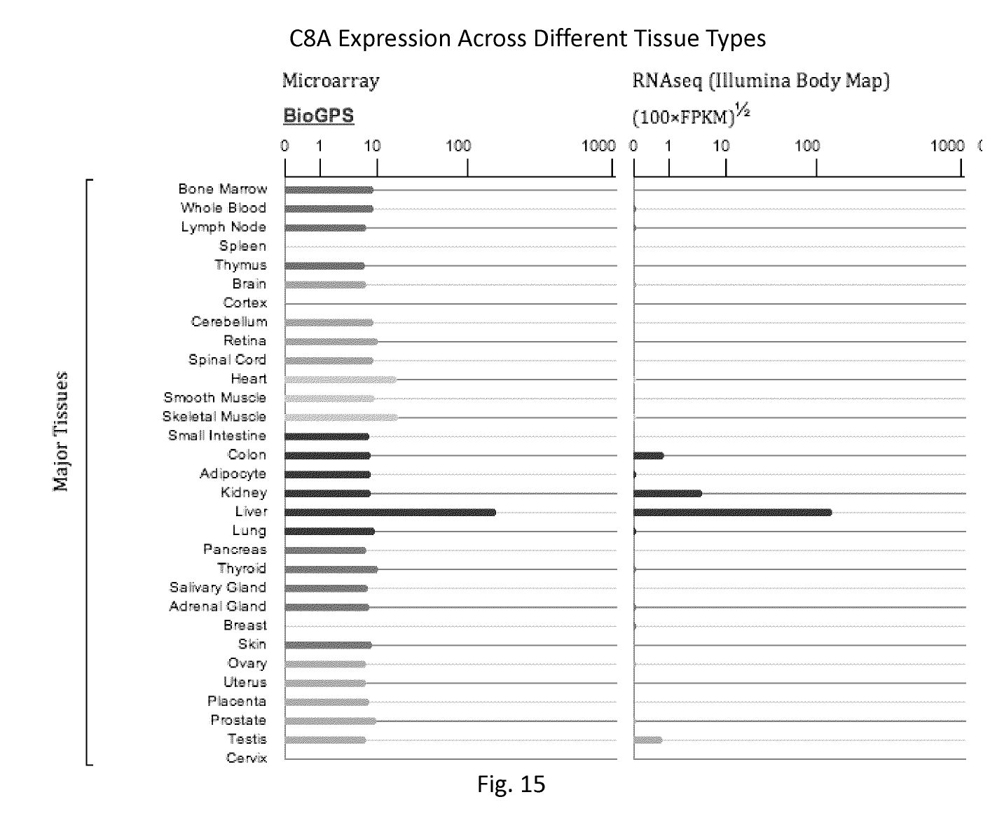

FIG. 16 shows the C9 expression across different tissue types.

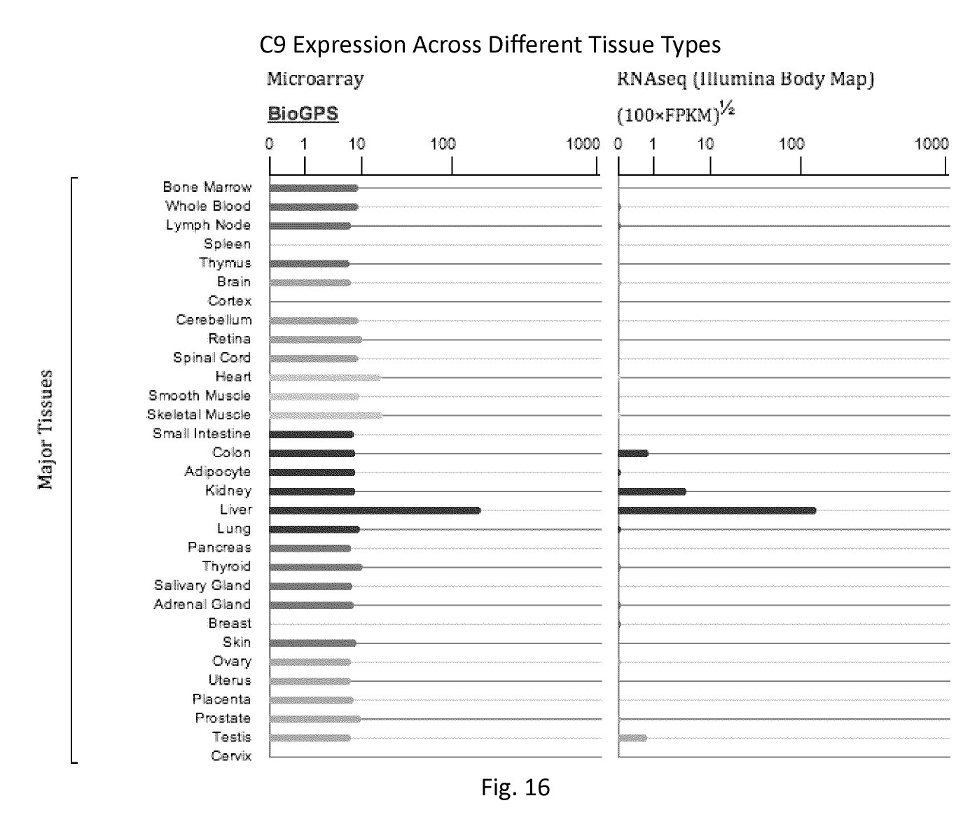

FIG. 17 shows the C9 expression across different tissue types.

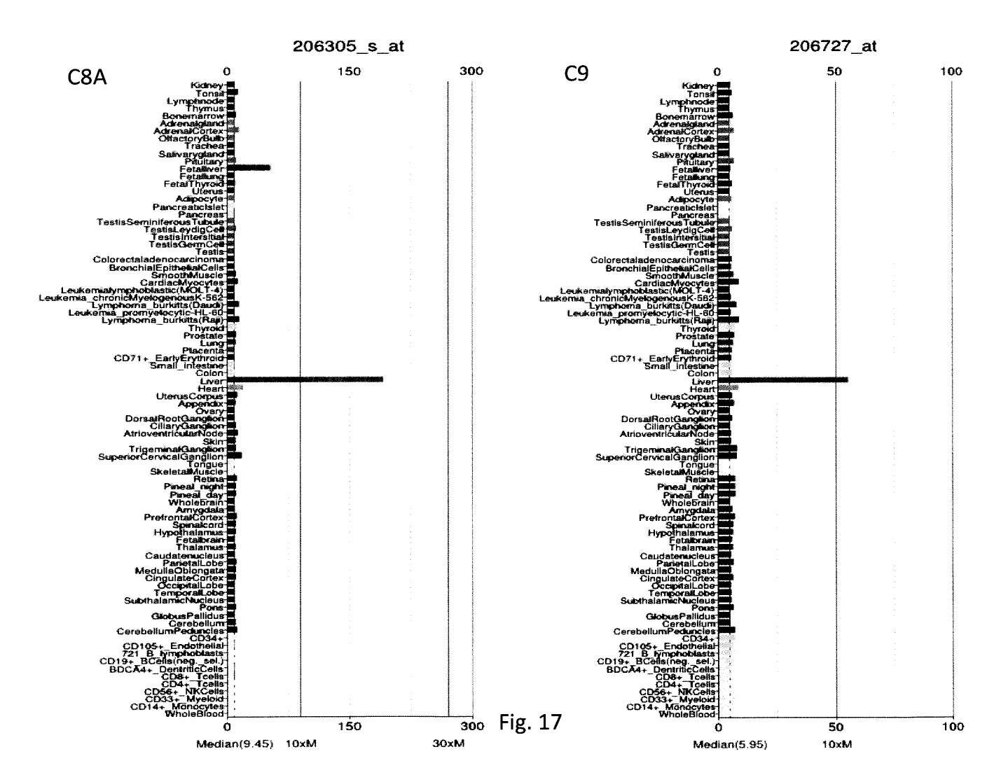

FIG. 18 shows the C8A and C9 expression in TCGA HER2+ cohort; (A) shows alteration of gene expression caused by gene amplification or mRNA upregulation of C8A (17%) and C9 (2%), (B) show reverse phase protein array (RPPA) score for ESR1 antibody in altered and unaltered sample, (C) show reverse phase protein array (RPPA) score for BCL2 antibody in altered and unaltered sample and (D) show reverse phase protein array (RPPA) score for CDKN1B antibody in altered and unaltered sample.

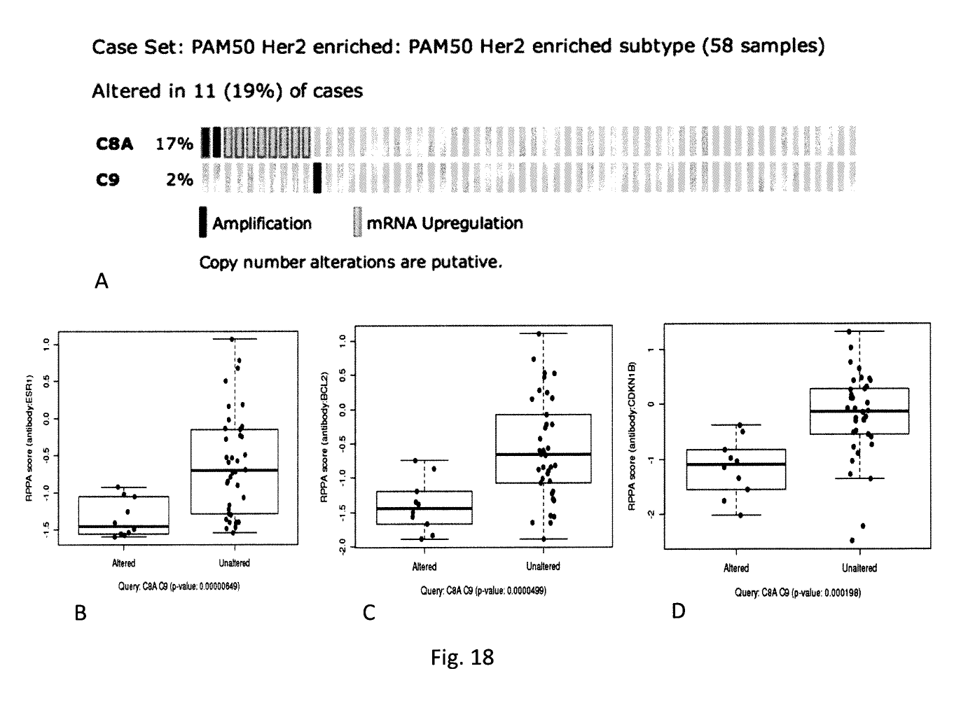

FIG. 19 shows the Kaplan Meier Survival Analysis for DFS [OR56A1] comparing observation vs treatment for (A) low and (B) high expression

FIG. 20 shows the Kaplan Meier Survival Analysis for DFS [PRR20C] comparing observation vs treatment for (A) low and (B) high expression

FIG. 21 shows the promoter analysis of [C8A, OR56A1, PRR20C] indicating that Serum Response Factor (SRF) as a transcription factor responsible for differential expression of C8A, OR56A1 and PRR20C.

FIG. 22 shows the top 20 predictive single gene biomarkers from the exploratory analysis of the TransHERA cohort

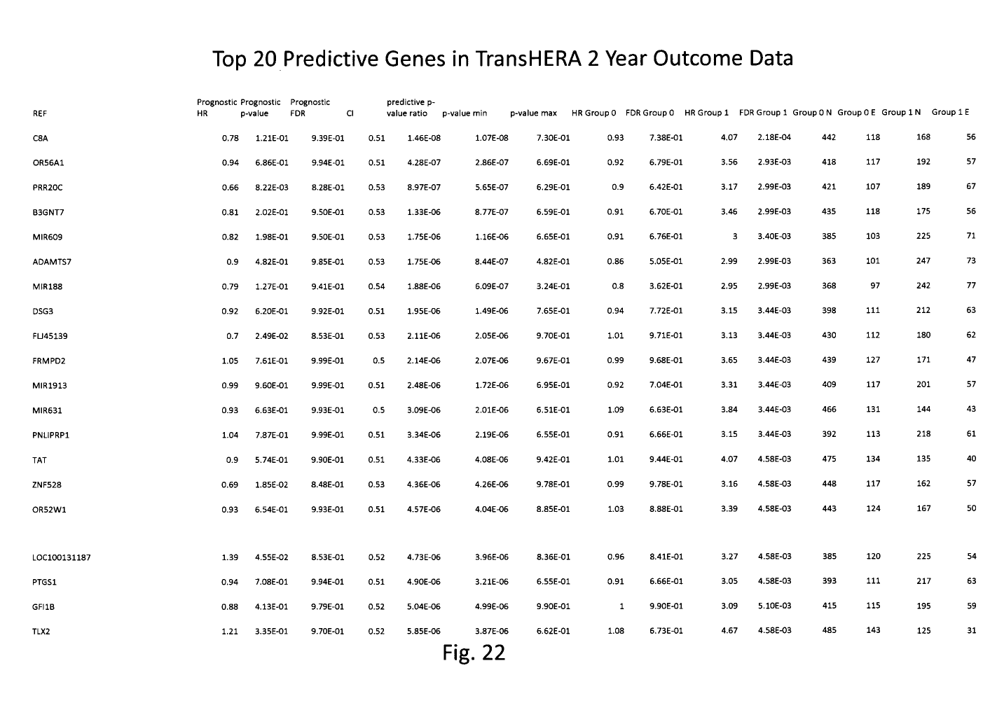

FIG. 23 shows the Kaplan Meier Survival Analysis for DFS [FRMPD2, PTGS1, OR52W1, MIR631, DSG3] comparing observation vs treatment for (A) low and (B) high expression.

FIG. 24 shows the promoter analysis of [FRMPD2, PTGS1, OR52W1, MIR631, DSG3].

FIG. 25 shows the Kaplan Meier Survival Analysis for DFS [TAT, MIR1913, PNLIPRP1, ADAMTS7, MIR188] comparing observation vs treatment for (A) low and (B) high expression.

FIG. 26 shows the promoter analysis of [TAT, PNLIPRP1, ADAMTS7].

FIG. 27 shows the Kaplan Meier Survival Analysis for DFS [OR52W1, B3GNT7, OR56A1] comparing observation vs treatment for (A) low and (B) high expression.

FIG. 28 shows the promoter analysis of [OR52W1, B3GNT7, OR56A1].

### DETAILED DESCRIPTION

In embodiments there is disclosed a method for prognosis of breast cancer outcome and/or predicting the benefit of treatment with an anti-ErbB antibody, comprising the step of:
(a) providing information on breast cancer treatment based on a comparison of mRNA expression level of a gene signature consisting of at least one biomarker chosen from C8A, OR56A1, PRR20C, B3GNT7, MIR609, ADAMTS7, MIR188, DSG3, FLJ45139, FRMPD2, MIR1913, MIR631, PNLIPRP1, TAT, ZNF528, OR52W1, LOC100131187, PTGS1, GFI1B, and TLX2 in a test sample from a subject, to a reference expression level of the biomarker,
   wherein the biomarker expression level is quantified using a biological assay;
   wherein over expression of the identified biomarker in the test sample indicates a poor breast cancer outcome and a beneficial treatment with the anti-ErbB antibody, and
   wherein minimal expression, or under expression of the biomarker in the test sample indicates positive outcome and a lack of benefit of treatment with the anti-ErbB antibody.

According to another embodiment, there is disclosed a method for treatment of cancer in an individual comprising:
a) determining, in a tumor sample from the patient, a value for at least one biomarker chosen from C8A, OR56A1, PRR20C, B3GNT7, MIR609, ADAMTS7, MIR188, DSG3, FLJ45139, FRMPD2, MIR1913, MIR631, PNLIPRP1, TAT, ZNF528, OR52W1, LOC100131187, PTGS1, GFI1B, and TLX2;
b) comparing the value obtained in step a) for the biological marker with a predetermined reference value for the same biomarker; and
c) administering a therapeutic agent to the individual if the value of the biomarker is above/below the predetermined reference value.

According to another embodiment, there is disclosed a method for treatment of cancer in an individual comprising:
a) detecting, in a tumor sample from the patient, at least one biomarker chosen from C8A, OR56A1, PRR20C, B3GNT7, MIR609, ADAMTS7, MIR188, DSG3, FLJ45139, FRMPD2, MIR1913, MIR631, PNLIPRP1, TAT, ZNF528, OR52W1, LOC100131187, PTGS1, GFI1B, and TLX2; and
b) administering a therapeutic agent to the individual if the at least one biomarker is detected in the tumor sample.

According to another embodiment, there is disclosed a method of identifying a cancer patient suitable for treatment with a therapeutic agent, comprising:
a) determining, in a tumor sample from the patient, a value for at least one biomarker chosen from C8A, OR56A1, PRR20C, B3GNT7, MIR609, ADAMTS7, MIR188, DSG3, FLJ45139, FRMPD2, MIR1913, MIR631, PNLIPRP1, TAT, ZNF528, OR52W1, LOC100131187, PTGS1, GFI1B, and TLX2;
b) comparing the value obtained in step a) for the biological marker with a predetermined reference value for the same biomarker; and
c) identifying the cancer patient as suitable for treatment with the therapeutic agent if the value of the biomarker is above/below the predetermined reference value.

According to another embodiment, there is disclosed a method of identifying a cancer patient suitable for treatment with a therapeutic agent, comprising:
a) detecting, in a tumor sample from the patient, at least one biomarker chosen from C8A, OR56A1, PRR20C, B3GNT7, MIR609, ADAMTS7, MIR188, DSG3, FLJ45139, FRMPD2, MIR1913, MIR631, PNLIPRP1, TAT, ZNF528, OR52W1, LOC100131187, PTGS1, GFI1B, and TLX2; and
b) identifying the cancer patient as suitable for treatment with the therapeutic agent if the at least one biomarker is detected in the tumor sample.

In one specific embodiment, the step of “identifying the cancer patient as suitable for treatment with the therapeutic agent” comprises identifying or selecting the patient as more likely to respond to a therapy with the therapeutic agent and further comprises selecting the therapy comprising the therapeutic agent.

According to another embodiment, there is disclosed a method for the prognosis of cancer, comprising:
a) determining, in a tumor sample from the patient, a value for at least one biomarker chosen from C8A, OR56A1, PRR20C, B3GNT7, MIR609, ADAMTS7, MIR188, DSG3, FLJ45139, FRMPD2, MIR1913, MIR631, PNLIPRP1, TAT, ZNF528, OR52W1, LOC100131187, PTGS1, GFI1B, and TLX2; and
b) comparing the value obtained in step a) for the biomarker with a predetermined reference value for the same biological marker, which predetermined reference value is correlated with a specific prognosis of cancer;
   wherein when the value of the biomarker is above the predetermined reference value, prognosis of the cancer outcome is a bad prognosis; and
   wherein when the value of the biomarker is below the predetermined reference value, prognosis of the cancer outcome is a good prognosis.

According to another embodiment, there is disclosed a method for the prognosis of cancer, comprising:
a) detecting, in a tumor sample from the patient, at least one biomarker chosen from C8A, OR56A1, PRR20C, B3GNT7, MIR609, ADAMTS7, MIR188, DSG3, FLJ45139, FRMPD2, MIR1913, MIR631, PNLIPRP1, TAT, ZNF528, OR52W1, LOC100131187, PTGS1, GFI1B, and TLX2; and
b) identifying the cancer as a cancer with a bad prognosis, when the at least one biomarker is detected in the tumor sample.

As used herein, the terms “biomarker” or “biological marker” is intended to mean a measurable indicator of the level of a gene or a gene product thereof, and includes gene copy number, mRNA level, protein level, or the level of a peptide which is associated with a protein of interest.

In specific embodiments, the method for treatment of cancer in an individual, the method of identifying a cancer patient suitable for treatment with a therapeutic agent, and the method for the prognosis of cancer are in vitro methods. In such in vitro methods, the value for the at least one biomarker is determined or detected in a sample obtained from an individual, for example, a patient, i.e., without including an active surgical step carried out on the human body.

According to an embodiment, the at least one biomarker chosen from C8A, OR56A1, PRR20C, B3GNT7, MIR609, ADAMTS7, MIR188, DSG3, FLJ45139, FRMPD2, MIR1913, MIR631, PNLIPRP1, TAT, ZNF528, OR52W1, LOC100131187, PTGS1, GFI1B, and TLX2. According to a preferred embodiment, the biomarker is C8A. According to another embodiment, the biomarker a combination of C8A, OR56A1 and PRR20C.

According to an embodiment, the cancer is breast cancer.

The methods of the present invention require the determination of a reference value for the at least one biomarker.

According to a first illustrative embodiment, a reference value for the biological marker may be predetermined by carrying out a method comprising the steps of:
a) providing at least one collection of tumor samples selected from the group consisting of:
   i) a collection of tumor samples from cancer patients with a known cancer classification, having undergone anti-cancer treatment, and subsequently having no cancer relapse or no cancer recurrence after the anti-cancer treatment;
   ii) a collection of tumor samples from cancer patients with a known cancer classification, having undergone anti-cancer treatment, and subsequently having cancer relapses or recurrences after the anti-cancer treatment.
b) quantifying, for each sample comprised in a collection of tumor samples provided at step a), the said biological marker, whereby a collection of quantification values for the said biological marker and for the said collection of tumor samples is obtained;
c) calculating, from the said collection of quantification values obtained at the end of step b), the mean quantification value for the said biological marker, whereby a predetermined reference value for said biological marker that is correlated with a specific cancer prognosis is obtained.

According to the method for obtaining predetermined reference values above, more than one predetermined reference value may be obtained for a single biological marker. For example, for a single biological marker, the method above allows the determination of at least four predetermined reference values for the same biological marker, respectively one predetermined reference value calculated from the mean quantification value obtained when starting, at step a), with each of the collections (i) and (ii) of tumor samples that are described above.

According to a second illustrative embodiment, reference values used for comparison at step b) of the method may also consist of “cut-off” values that may be determined as described hereunder.

Each reference (“cut-off”) value for each biological marker may be predetermined by carrying out a method comprising the steps of:
a) selecting a biological marker for which a reference value is to be determined;
b) providing a collection of tumor tissue samples from cancer patients;
c) providing, for each tumor sample provided at step b), information relating to the actual clinical outcome for the corresponding cancer patient;
d) providing a serial of arbitrary quantification values for the said biological marker selected at step a);
e) quantifying the said biological marker in each tumor tissue sample contained in the collection provided at step b);
f) classifying the said tumor samples in two groups for one specific arbitrary quantification value provided at step c), respectively:
   (i) a first group comprising tumor samples that exhibit a quantification value for the said marker that is lower than the said arbitrary quantification value contained in the said serial of quantification values;
   (ii) a second group comprising tumor samples that exhibit a quantification value for the said marker that is higher than the said arbitrary quantification value contained in the said serial of quantification values;
   whereby two groups of tumor samples are obtained for the said specific quantification value, wherein the tumors samples of each group are separately enumerated;
g) calculating the statistical significance between (i) the quantification value for the said biological marker obtained at step e) and (ii) the actual clinical outcome of the patients from which tumor samples contained in the first and second groups defined at step f) derive;
h) reiterating steps f) and g) until every arbitrary quantification value provided at step d) is tested;
i) setting the said reference value (“cut-off” value) as consisting of the arbitrary quantification value for which the highest statistical significance (most significant) has been calculated at step g).

As it is disclosed above, the said method allows the setting of a single “cut-off” value permitting discrimination between bad and good outcome prognosis or between patients that are suitable for a treatment and those which are not suitable for a treatment. Practically, high statistical significance values (e.g. low P values) are generally obtained for a range of successive arbitrary quantification values, and not only for a single arbitrary quantification value. Thus, in one alternative embodiment of the method of determining “cut-off” values above, a minimal statistical significance value (minimal threshold of significance, e.g. maximal threshold P value) is arbitrarily set and the range of arbitrary quantification values for which the statistical significance value calculated at step g) is higher (more significant, e.g. lower P value) are retained, whereby a range of quantification values is provided. The said range of quantification values consist of a “cut-off” value according to the invention. According to this specific embodiment of a “cut-off” value, bad or good clinical outcome prognosis can be determined, or patients suitable for treatment can be identified, by comparing the value obtained for the biological marker in the tumor sample with the range of values delimiting the said “cut-off” value, for one specific biological marker. In certain embodiments, a cut-off value consisting of a range of quantification values for the considered biological marker, consists of a range of values centered on the quantification value for which the highest statistical significance value is found (e.g. generally the minimum P value which is found).

In certain preferred embodiments of step c) of the method for determining cut-off values above, the said information relating to the actual clinical outcome of the patients are selected from the group consisting of (i) the duration of the disease-free survival (DFS) and (ii) the overall survival (OS).

In certain embodiments, the reference predetermined value consists of a “cut-off” value, as already disclosed above, which “cut-off” value consists of a median quantification value for the biological marker of interest that discriminates between bad cancer prognosis and good cancer prognosis.

According to a third illustrative embodiment, in embodiments wherein the biological marker consists of the expression level of a gene, the predetermined reference value may consist of the gene expression value that correlates with bad cancer prognosis, e.g. relapses or recurrences, short survival time, etc., or in contrast may consist of the gene expression value that correlates with good cancer prognosis, e.g. no metastasis at all or long disease-free survival time. The gene expression value may be expressed as any arbitrary unit. For instance, the gene expression value may be expressed as the difference (deltaCT) between (i) the amount of the biological marker-specific mRNA and (ii) the amount of an unrelated mRNA, found in the tumor sample, such as for example the ribosomal 18S mRNA. Illustratively, for human breast cancer, the difference between (i) the amount of the biological marker specific mRNA and (ii) the amount of an unrelated mRNA may be arbitrarily assigned to consist of the deltaCT and of the mean of all values from the reference group (e.g. for patients undergoing early steps of metastasis processes and relapses, set to “100%”). In these embodiments, the quantification value generated for a particular gene-specific mRNA is more than 100%, then a better cancer prognosis than with the predetermined reference value is obtained.

In embodiments, when the value of the biomarker is above the predetermined reference value, the therapeutic agent is an anti-ErbB antibody. Alternatively, when the value of the biomarker is below the predetermined reference value, the therapeutic agent is different than an anti-ErbB antibody. According to an embodiment, the anti-ErbB antibody may also be administered in combination with another therapeutic agent.

A variety of anti-ErbB antibodies are known in the prior art, and a variety of these antibodies are anti-HER2 antibodies. Anti-ErbB antibody, and anti-HER2 antibodies are preferably monoclonal antibodies. They may either be so-called chimeric antibodies, humanized antibodies or fully human antibodies. They may either be full length anti-ErbB/anti-HER2 antibodies; anti-ErbB/anti-HER2 antibody fragments having the same biological activity; including amino acid sequence variants and/or glycosylation variants of such antibodies or fragments. Examples of humanized anti-HER2 antibodies are known under the INN names Trastuzumab and Pertuzumab. Another suitable anti-HER2 antibody is T-DM1, which is an antibody-toxin conjugate consisting of huMAb4D5-8 (HERCEPTIN™) and a maytansinoide (viz. DM1=N2′-deacetyl-N2′-(3-mercapto-1-oxopropyl)-maytansine; a highly potent antimicrotubule agent) which conjugate (with a MCC linker) is currently under development for metastatic breast cancer. Other HER2 antibodies with various properties have been described in Tagliabue et al., Int. J. Cancer, 47:933-937 (1991); McKenzie et al., Oncogene, 4:543-548 (1989); Cancer Res., 51:5361-5369 (1991); Bacus et al., Molecular Carcinogenesis, 3:350-362 (1990); Stancovski et al., PNAS (USA), 88:8691-8695 (1991); Bacus et al., Cancer Research, 52:2580-2589 (1992); Xu et al., Int. J. Cancer, 53:401-408 (1993); WO94/00136; Kasprzyk et al., Cancer Research, 52:2771-2776 (1992); Hancock et al., Cancer Res., 51:4575-4580 (1991); Shawver et al., Cancer Res., 54:1367-1373 (1994); Arteaga et al., Cancer Res., 54:3758-3765 (1994); Harwerth et al., J. Biol. Chem., 267:15160-15167 (1992); U.S. Pat. No. 5,783,186; and Klapper et al., Oncogene, 14:2099-2109 (1997). The most successful therapeutic anti-HER2 antibody is Trastuzumab sold by Genentech Inc. and F. Hoffmann-La Roche Ltd under the trade name HERCEPTIN™. Further details on the HER2 antigen and antibodies directed thereto are described in many patent and non-patent publications (for a suitable overview see U.S. Pat. No. 5,821,337 and WO 2006/044908).

In one specific embodiment the anti-ErbB antibody is an anti-Her2 antibody. In one embodiment the anti-Her2 antibody is e.g. selected from the group of Trastuzumab, Pertuzumab and T-DM1 and may also consist of a mixture of anti-HER2 antibodies such as e.g. Trastuzumab and Pertuzumab or T-DM1 and Pertuzumab. It has been found that the combination of Pertuzumab and Trastuzumab is active and well tolerated in patients with metastatic HER2-positive breast cancer who had experienced progression during prior trastuzumab therapy [see e.g. Baselga, J. et al., Journal of Clin. Oncol. Vol 28 (7) 2010: pp. 1138-1144]. The terms “Trastuzumab”, “Pertuzumab” and “T-DM1” encompass all corresponding anti-HER2 antibodies that fulfill the requirements necessary for obtaining a marketing authorization as an identical or biosimilar product in a country or territory selected from the group of countries consisting of the USA, Europe and Japan. Trastuzumab has the CDR regions defined in EP-B-590058. Pertuzumab has the CDR regions defined in WO 01/00245. The activity of Trastuzumab in the BT-474 antiproliferation assay [Nahta, R. et al., “The HER-2-targeting antibodies Trastuzumab and Pertuzumab synergistically inhibit the survival of breast cancer cells”, Cancer Res. 2004; 64:2343.2346] has been found to be between 0.7-1.3×10⁴ U/mg. T-DM1 is described in WO 2005/117986.

Examples of therapeutic agents that may be used in the treatment of breast cancer in combination with the anti-ErbB antibody, or separately from the anti-ErbB antibody (that is, as a therapeutic agent different than an anti-ErbB antibody) include doxorubicin (Adriamycin®), Liposomal doxorubicin (Doxil®), epirubicin (Ellence®), taxanes such as paclitaxel (Taxol®), docetaxel (Taxotere®), Albumin-bound paclitaxel (nab-paclitaxel or Abraxane®), fluorouracil (5-FU), cyclophosphamide (Cytoxan®), and platinum agents such as cisplatin and carboplatin, Vinorelbine (Navelbine®), Capecitabine (Xeloda®), Gemcitabine (Gemzar®), Mitoxantrone; Ixabepilone (Ixempra®), Eribulin (Halaven®), alone or in combinations thereof.

The determination of the value for a biological marker is with a biological assay. In such biological assays, the tumor sample is, for example, contacted with an agent that specifically binds to the biomarker, thereby forming a complex between the agent and biomarker which is detectable. In biological assays, the biomarker present in the tumor sample may also be amplified and the amplified biomarker may be detected with an agent that specifically binds to the amplified biomarker. Based on the detection, the value of the biological marker is determined.

According to an embodiment, the value for a biological marker is an expression level, and determining the expression level for the biomarker comprises contacting the tumor sample with an agent that specifically binds to the biomarker, thereby forming a complex between the agent and biomarker, detecting the amount of complex formed, thereby measuring the expression level of the biomarker.

According to another embodiment, the value for a biological marker is an expression level, and determining the expression level for the biomarker comprises amplifying the biomarker present in the sample and detecting the amplified biomarker with an agent that specifically binds to the amplified biomarker, thereby measuring the level of biomarker.

According to another embodiment, there is disclosed a kit for the prognosis of breast cancer outcome and/or predicting the benefit of treatment with an anti-ErbB antibody. The kit comprises (a) an agent that specifically detects a biomarker, wherein the biomarker is at least one biomarker chosen from C8A, OR56A1, PRR20C, B3GNT7, MIR609, ADAMTS7, MIR188, DSG3, FLJ45139, FRMPD2, MIR1913, MIR631, PNLIPRP1, TAT, ZNF528, OR52W1, LOC100131187, PTGS1, GFI1B, and TLX2; and (b) instructions to use the kit.

According to another embodiment, there is disclosed a kit for identifying a cancer patient suitable for treatment with a therapeutic agent. The kit comprises (a) an agent that specifically detects a biomarker, wherein the biomarker is at least one biomarker chosen from C8A, OR56A1, PRR20C, B3GNT7, MIR609, ADAMTS7, MIR188, DSG3, FLJ45139, FRMPD2, MIR1913, MIR631, PNLIPRP1, TAT, ZNF528, OR52W1, LOC100131187, PTGS1, GFI1B, and TLX2; and (b) instructions to use the kit.

According to another embodiment, there is disclosed a kit for the prognosis of cancer. The kit comprises (a) an agent that specifically detects a biomarker, wherein the biomarker is at least one biomarker chosen from C8A, OR56A1, PRR20C, B3GNT7, MIR609, ADAMTS7, MIR188, DSG3, FLJ45139, FRMPD2, MIR1913, MIR631, PNLIPRP1, TAT, ZNF528, OR52W1, LOC100131187, PTGS1, GFI1B, and TLX2; and (b) instructions to use the kit.

Examples for an agent that specifically binds to the biomarker are nucleic acid probes (for example a pair of nucleic acid probes) or protein binding agents such as antibodies, antibody fragments, or aptamers, or combinations thereof.

In one specific embodiment, the at least one biomarker is amplified with a PCR based method.

According to an embodiment, the expression level is one of an mRNA expression level, a protein expression level, or combinations thereof. According to another embodiment, the value for a biological marker is a gene copy number.

Suitable biological assays include but are not limited to PCR based methods, hybridization based methods, sequencing methods, protein detection methods, or combinations thereof. Examples of suitable PCR based methods comprise any form of reverse transcriptase polymerase chain reaction (RT-PCR), and any form of quantitative reverse transcriptase polymerase chain reaction (QRT-PCR). Examples of suitable hybridization methods comprise nuclease protection assay, Northern blot analysis, in situ hybridization, and microarray based analysis based on any suitable arrays such as Affymetrix GeneChip™, Illumina™ DASL™ arrays, printed cDNA arrays, and the likes. Suitable sequencing methods comprise next generation sequencing (NGS) technologies. Suitable protein detecting methods comprises a Western blot analysis, an enzyme-linked immunosorbent assay (ELISA), immunohistochemistry analysis, an immunoprecipitation followed by an SDS-PAGE analysis, a proteomics analysis, such as a quantitative proteomics analysis.

According to an embodiment, the cancer is breast cancer. In embodiments, when the value of the biomarker is above the predetermined reference value, the therapeutic agent is an anti-ErbB antibody. Also, when the value of the biomarker is below the predetermined reference value, the therapeutic agent is different than an anti-ErbB antibody.

According to another embodiment, when the value of the biomarker is above the predetermined reference value, the bad prognosis is indicative of a beneficial treatment with an anti-ErbB antibody. Also, when the value of the biomarker is below the predetermined reference value the good prognosis is indicative of a non-beneficial treatment with an anti-ErbB antibody.

C8A is a member of the membrane attack complex (MAC) and is part of the innate immune system. C8A inserts into the membrane of the target cell and binds with multiple copies of the pore-forming C9.

The methods of the present invention use test samples. Test samples include but are not limited to normal and tumor tissue samples, such as for example formalin-fixed, paraffin-embedded (FFPE) or frozen tissue samples. Also included are biological fluid samples, such as samples from blood, blood plasma, lymph, saliva, cerebrospinal fluid, pericardial fluid, peritoneal fluid, pleural fluid, and urine. Also included in the test samples are the DNA and RNA which may be extracted and isolated therefore. According to a preferred embodiment, the samples are formalin-fixed, paraffin-embedded (FFPE) samples.

The TransHERA trial has been designed to test the anti-HER2 therapy trastuzumab given for 1 or 2 years after standard chemotherapy for improvements in disease-free survival (DFS) and overall survival as compared to an observation arm in patients with over-expression of the HER-2 protein. In total 5,102 patients have been recruited from December 2001 to June 2005 and the results have changed the treatment of HER2 positive breast cancer.

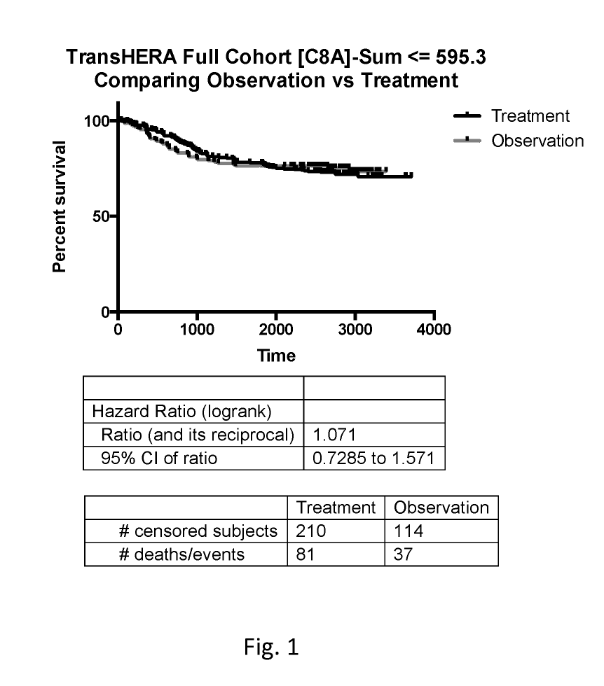

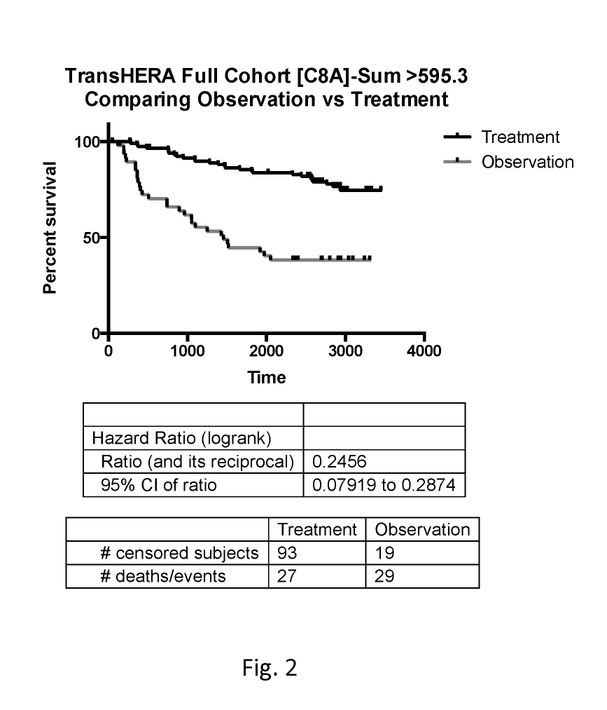

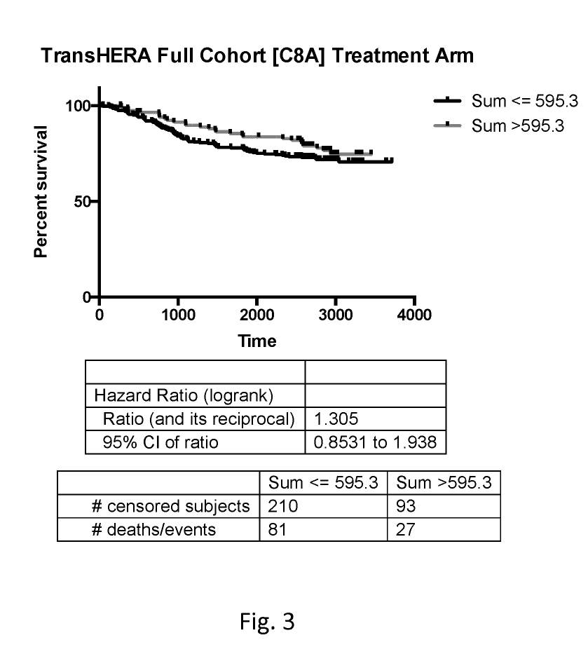

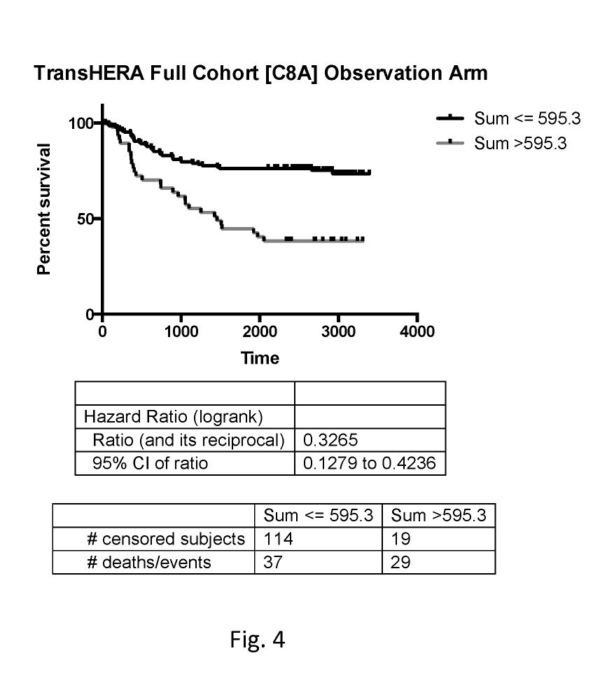

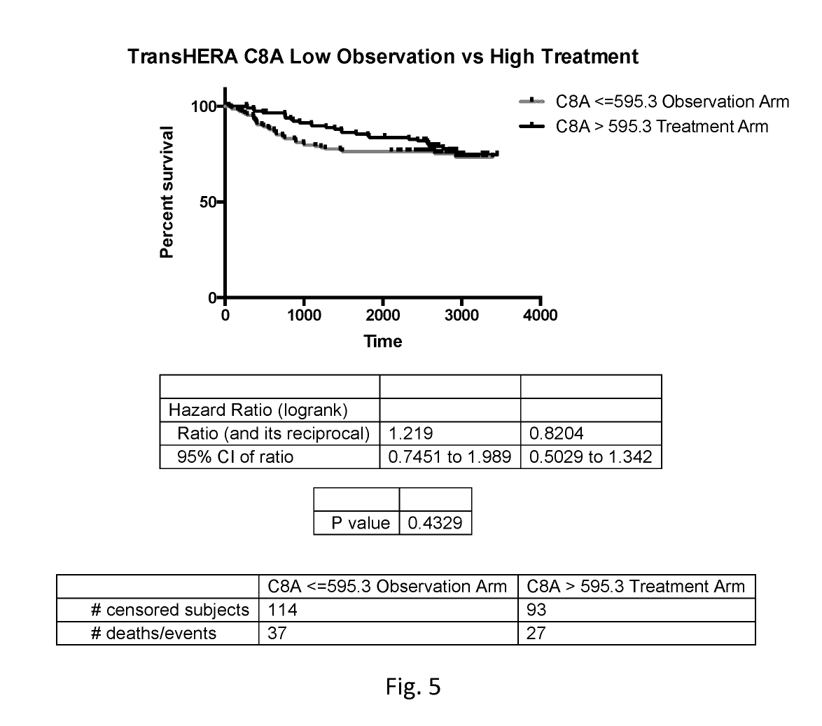

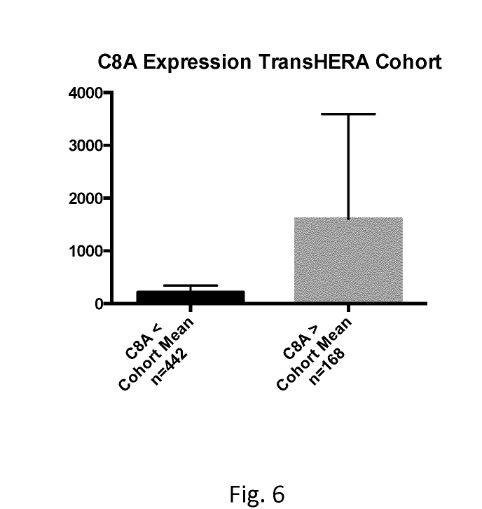

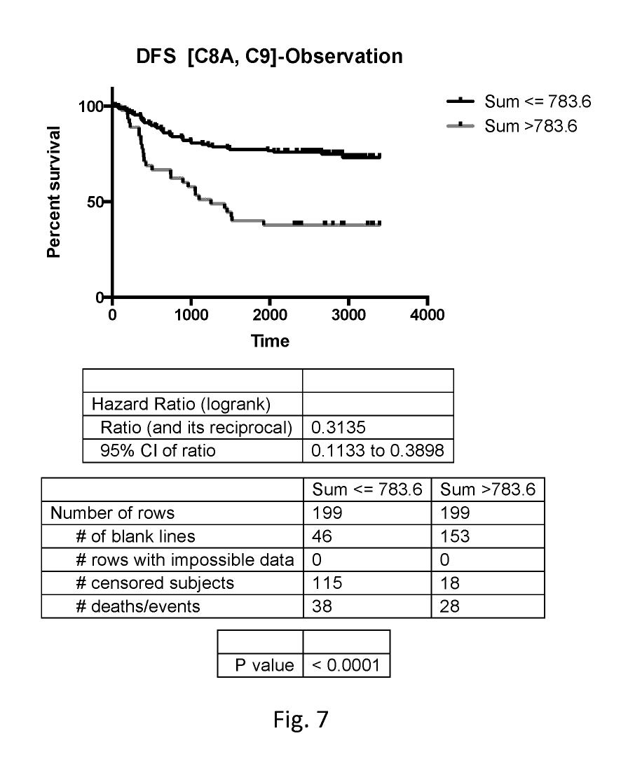

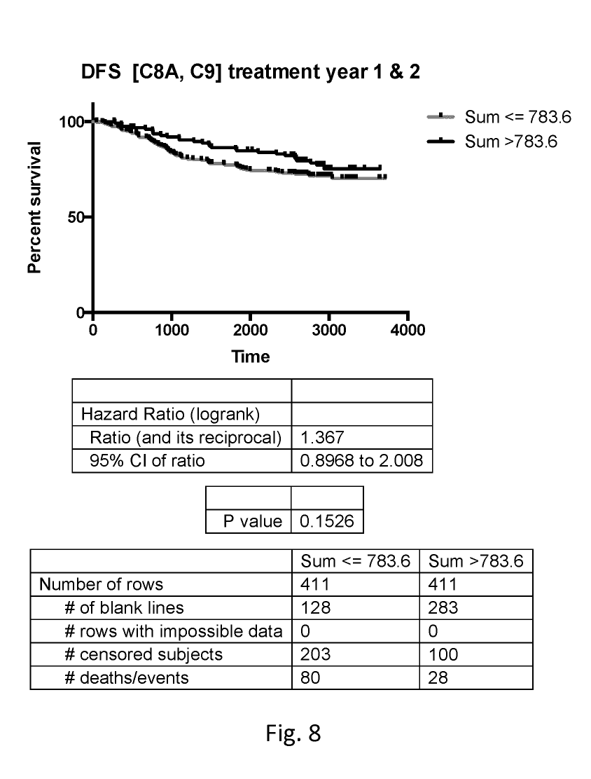

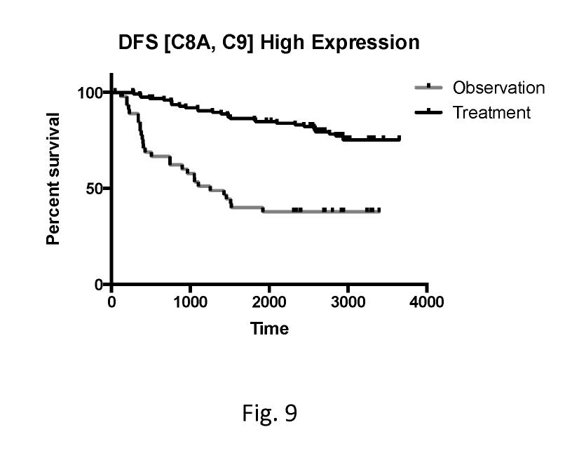

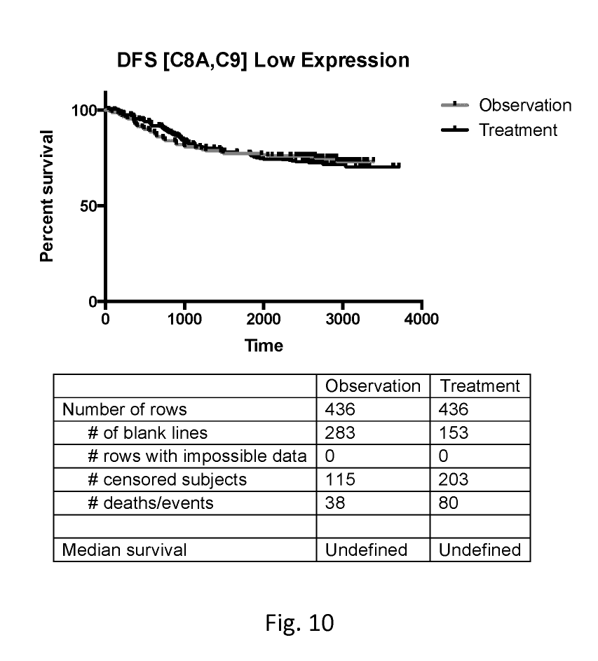

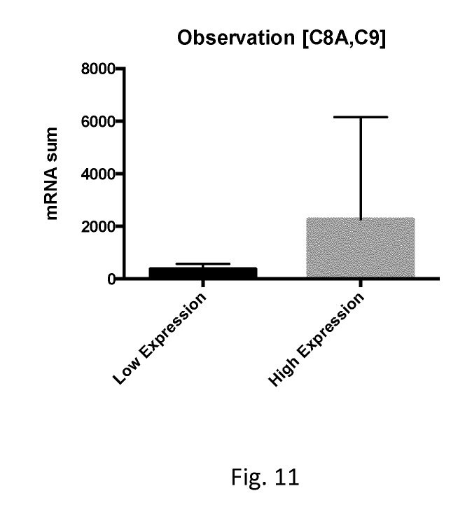

According to an embodiment, to determine potential prognostic and/or predictive biomarkers, an exploratory analysis is conducted using mRNA expression data from 610 TransHERA formalin-fixed, paraffin-embedded (FFPE) clinical trial samples profiled on the IIlumina™ Whole-Genome DASL™ platform. The observation arm consisted of 199 samples with 66 DFS events (e) and the treatment arm included 411 samples with treatment of trastuzumab (an anti-ErbB antibody) for one year and two year with 108 DFS events. Now referring to FIGS. 1 to 11, the exploratory analysis using DFS as the endpoint identified that samples with expression of C8A greater than cohort mean (high expression) in the observation arm indicated poor outcome (FIG. 4; HR=0.33 95% CI:0.13-0.42) suggesting C8A is a prognostic biomarker. When comparing the observation samples (n=151 e=37) and treatment samples (n=291 e=81) where C8A expression is lower than the cohort mean (low expression) shows no benefit of treatment (FIG. 1; HR=1.07 95% CI:0.72-1.57). Comparing the observation samples (n=48 e=29) and treatment samples (n=120 e=27) with high expression of C8A indicates a substantial benefit of treatment (FIG. 2; HR=0.246 95% CI=0.08-0.29) suggesting that C8A is also predictive of benefit for treatment with trastuzumab. Comparing the C8A low expression samples in the observation arm (n=151 e=37) against the C8A high expression samples (n=120 e=27) in the treatment arm shows no statistical difference in outcome (FIG. 5; log rank p-value=0.43). This suggests that the DFS outcome of HER2+ patients with low expression of C8A and no trastuzumab treatment is comparable to HER2+ patients with high expression of C8A that benefit from treatment with trastuzumab.

For the low expression group the average ratio of C8A/C9 is 2.3 and in the high expression group is 14.6 indicating an over expression of C8A when compared to C9. Reviewing mRNA expression levels of C8A in the Cancer Cell Line Encyclopedia (n=1037) has a mean value=3.77 stdev=0.26 with min=3.34 and max=7.12. Examples with C8A over expression in cancer cell lines include HEPG2_LIVER=7.1, OV90_OVARY=6.7, DMS454_LUNG=6.9, JHH5_LIVER=5.6, HUH7_LIVER=5.3 and SNU719_STOMACH=5.7. From the GeneAtlas, a panel of 79 human tissues, C8A is highly expressed in liver tissue suggesting a fitness advantage for tumors with high expression of C8A.

TABLE 1
Expression values from Cancer Cell Line Encyclopedia

| | BT474 | SKBR3 | HCC1954 | UACC893 | MCF7 | All Cancer | | |
|---|---|---|---|---|---|---|---|---|
| Description | BREAST | BREAST | BREAST | BREAST | BREAST | Cell Average | Max | Min |
| C8A | 3.78 | 3.70 | 3.93 | 3.68 | 3.96 | 3.77 | 7.12 | 3.34 |
| C9 | 3.45 | 3.44 | 3.35 | 3.45 | 3.55 | 3.65 | 9.03 | 3.19 |
| C2 | 4.82 | 5.03 | 4.72 | 4.93 | 4.47 | 4.95 | 10.83 | 3.90 |
| C3 | 5.09 | 4.89 | 5.76 | 5.55 | 5.33 | 6.51 | 13.22 | 3.92 |
| C5 | 6.48 | 4.58 | 5.41 | 4.31 | 5.25 | 6.00 | 12.60 | 3.79 |
| C6 | 4.61 | 4.70 | 4.56 | 5.08 | 4.46 | 4.72 | 8.75 | 3.91 |
| ERBB2 | 11.78 | 12.02 | 12.63 | 13.48 | 7.91 | 6.98 | 13.52 | 5.32 |

From the five available HER2+ cell lines C8A does not appear to be differentially expressed and is near the minimum for 1000+ cell lines. Over expression of C8A is seen in approximately 25% of the TransHERA cohort. The max value of 7.12 is found in HEPG2_LIVER nearly a 2× increase on log scale. Other over expression of C8A cell lines (OV90_OVARY 6.7, DMS454_LUNG 6.9, JHH5_LIVER 5.6, HUH7_LIVER 5.3, SNU719_STOMACH 5.7). The max HER2 expression is OE19_OESOPHAGUS at 13.5. This data suggests that cancer cell lines do differentially express C8A, C9, C2, C3, C5, C6.

Identification of C8A as a single gene biomarker that is prognostic of DFS and predictive of benefit from trastuzumab has the potential to improve the standard of care in HER2+ breast cancer. Understanding the advantage of over expression of C8A related to the innate and adaptive immune response can give insight into the mechanisms that drive cancer.

### Lectin Pathway and the Membrane Attack Complex (MAC)

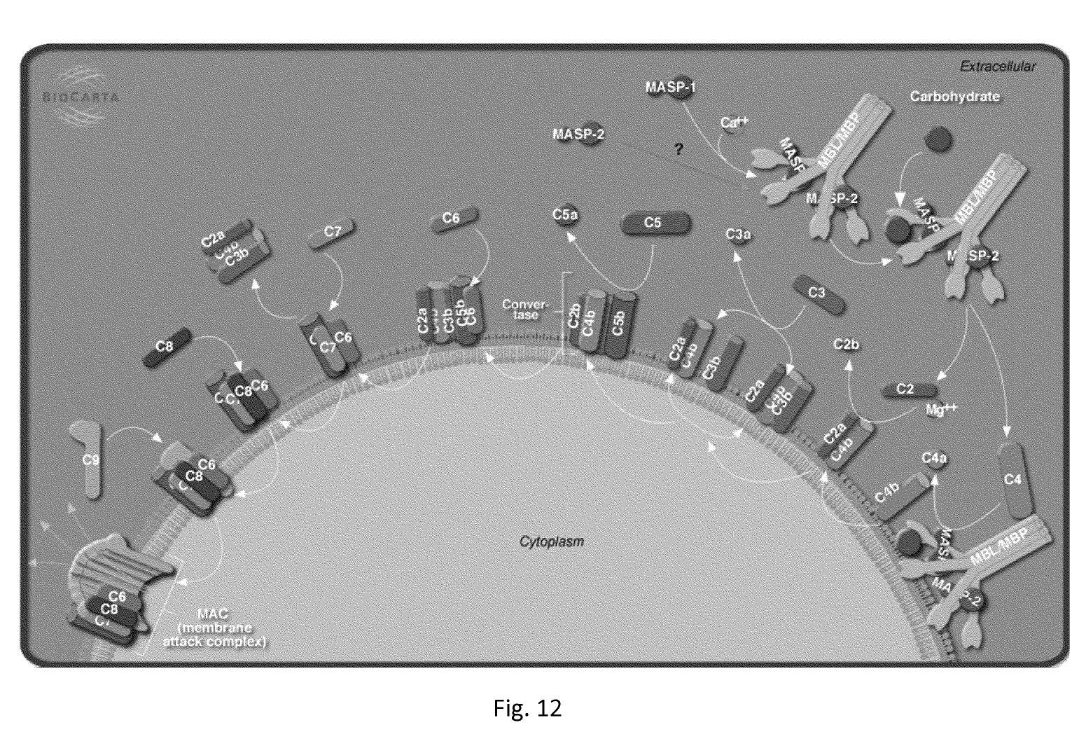

Now referring to FIG. 12. The complement cascade of proteolytic factors involved in cellular lysis can be initiated by several different factors, including antibody-dependent and antibody-independent recognition of infectious organisms. In the lectin-induced complement cascade, carbohydrates on the surface of microbial cells activate the complement cascade by binding to mannan-binding lectin (also called the mannan-binding protein, Mbl/Mbp). Mbp is an acute phase serum protein whose expression is induced by microbial infection. The binding of Mbl to microbial ligands activates the Mbl associated serine proteases Masp1 and Masp2, triggering the cleavage of C2 and C4 to create C4bC2a, a C3 convertase that cleaves large numbers of C3. Masp1 and Masp2 are similar to the C1 protease in the classical complement pathway. Once formed the C3 convertase cleaves and activates the remaining complement factors leading ultimately to formation of a pore in the bacterial membrane by the membrane attack complex (MAC) that lyses the bacterial cell. The lectin-induced pathway also appears to play an important role of the activation of phagocytotic cells by infection. Although the initiating event activating the complement cascade is distinct in the lectin-induced pathway, from the C3 convertase onward the lectin induced complement pathway is the same as the classical complement pathway. Since antibodies are not required in the lectin-induced pathway, this aspect of the immune response is part of the innate immune response. The importance of this pathway to the immune response has been demonstrated by the identification of children and adults with little or no Mbl who lacked normal phagocytotic responses and are highly susceptible to infection.

Constituent of the membrane attack complex (MAC) that plays a key role in the innate and adaptive immune response by forming pores in the plasma membrane of target cells. C8A inserts into the target membrane, but does not form pores by itself. Constituent of the membrane attack complex (MAC) that plays a key role in the innate and adaptive immune response by forming pores in the plasma membrane of target cells. C9 is the pore-forming subunit of the MAC. Component of the membrane attack complex (MAC). MAC assembly is initiated by proteolytic cleavage of C5 into C5a and C5b. C5b binds sequentially C6, C7, C8 and multiple copies of the pore-forming subunit C9. C8A is expressed at a higher ratio then C9. If higher expression of C8A is indicative of aggressive immune response then it would expected that high C8A would have favorable outcome. However, comparing the ratio of C8A/C9 for low expression group to C8A/C9 for high expression group it can be seen clearly that C8A/C9 ratio is much higher in the poor outcome group.

Exploring all combinations of the 20 genes (C8A, OR56A1, PRR20C, B3GNT7, MIR609, ADAMTS7, MIR188, DSG3, FLJ45139, FRMPD2, MIR1913, MIR631, PNLIPRP1, TAT, ZNF528, OR52W1, LOC100131187, PTGS1, GFI1B, and TLX2) where each gene signature is n<=5 genes can determine optimized gene signatures.

To determine biologically relevant optimal gene signatures, gene signatures that share a common biological feature (or attribute) are selected, such as sharing a common DNA promoter motif indicating a shared transcription factor responsible for differential expression of the gene signature, molecular GO annotation, canonical pathway, chromosomal location etc., which may indicate a biologically relevant gene signature. Numerous methods can be used to determine biomarkers or gene signatures of interest in a particular cohort that do not validate when tested in other cohorts.

By requiring gene signatures to have a common biological attribute such as a DNA promoter motif the biological relevance of the gene signature is indicated and reduces the likelihood that the gene signature results from over fitting or occurred by chance.

Therefore, according to another embodiment, the method of the present invention may further comprise step (b) when the gene signature comprises at least two biomarkers:
(b) optimizing the gene signature by identifying a common biological attribute between said at least two biomarker, to provide an optimized gene signature,
   wherein over expression in the test sample of the at least two biomarkers identified in said optimized gene signature indicates a poor breast cancer outcome and a beneficial treatment with ErbB antibodies, and
   wherein minimal expression, or under expression in the test sample of the at least two biomarkers identified in said optimized gene signature indicates positive outcome and a lack of benefit of treatment with ErbB antibodies.

The present invention will be more readily understood by referring to the following examples which are given to illustrate the invention rather than to limit its scope.

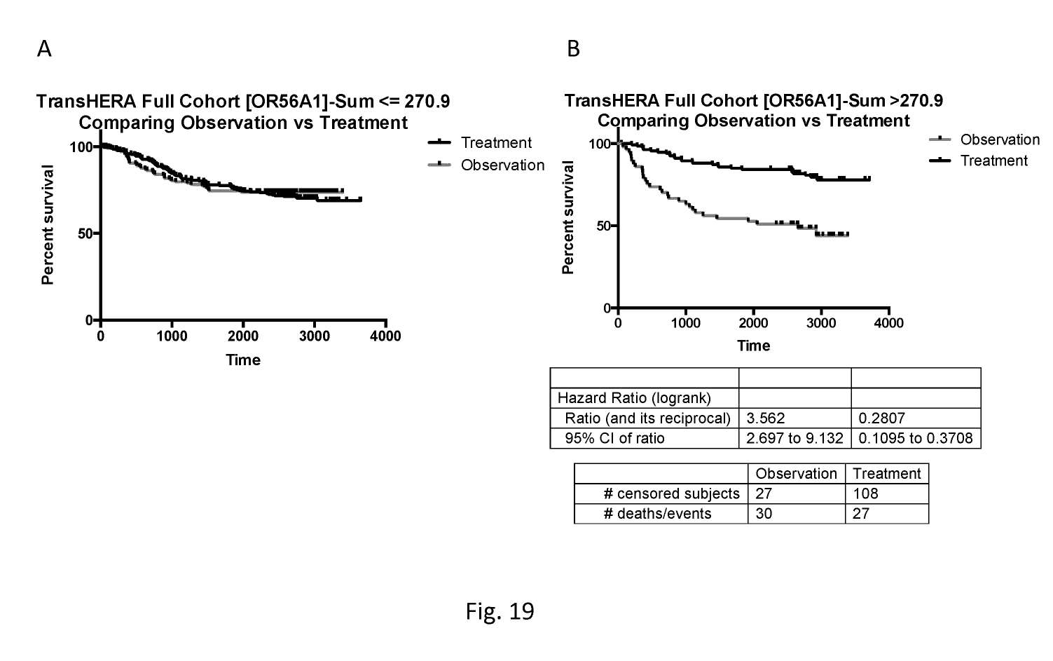

Additionally, the exploratory analysis indicates that over expression of [OR56A1] as compared to the cohort mean is predictive of benefit of treatment with an anti-ErbB antibody (FIG. 19B; HR=0.2807 95% CI=0.11-0.37) and no benefit of treatment for low or normal expression of [OR56A1] (FIG. 19A).

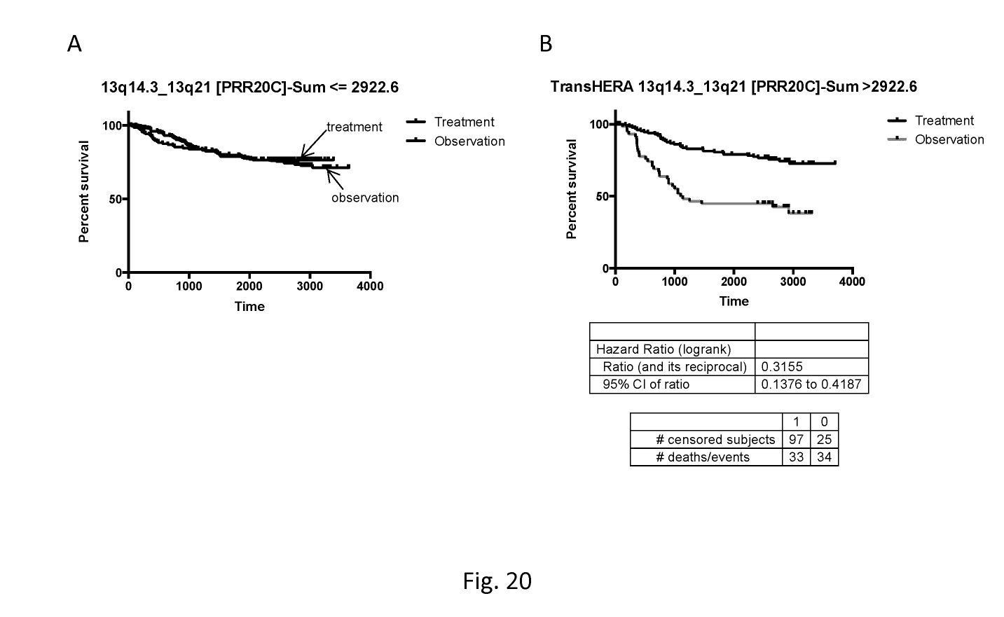

Additionally, the exploratory analysis indicates that over expression of [PRR20C] as compared to the cohort mean is predictive of benefit of treatment with an anti-ErbB antibody (FIG. 20B; HR=0.3155 95% CI=0.14-0.42) and no benefit of treatment for low or normal expression of [PRR20C] (FIG. 20A).

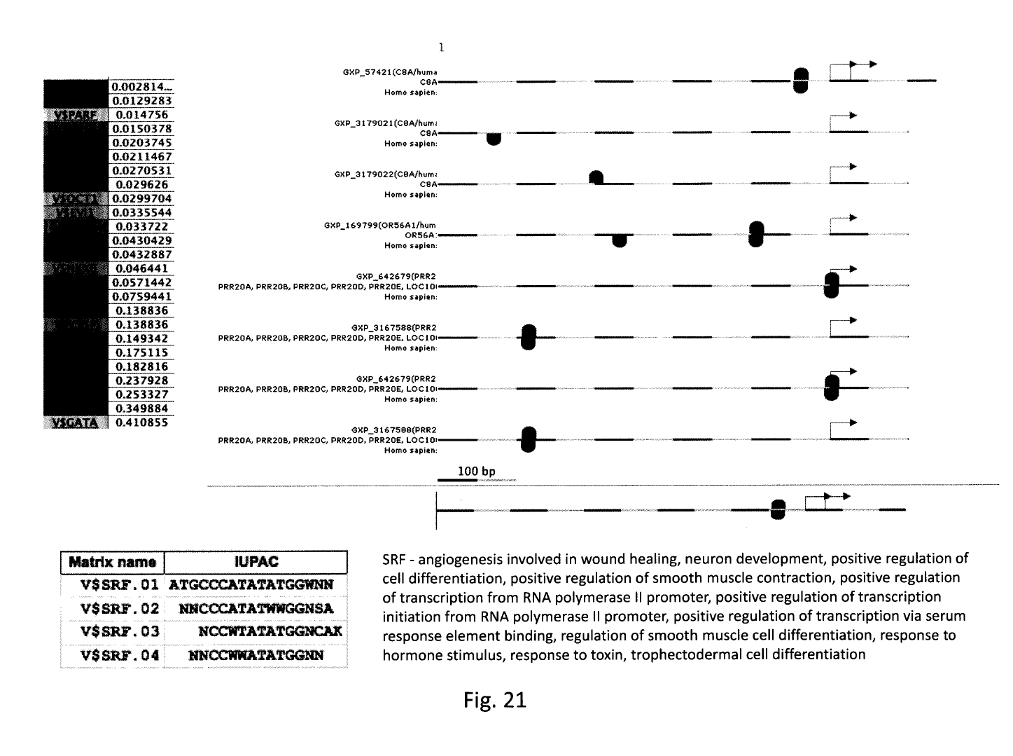

Promoter analysis of (FIG. 21) [C8A, OR56A1, PRR20C] indicates that the transcription factor Serum Response Factor (SRF) has promoter motifs in common with [C8A, OR56A1, PRR20C] p-value=0.003. This implicates SRF as the transcription factor responsible for differential expression of [C8A, OR56A1, PRR20C] and further supports the biological relevance of the three genes as independent biomarkers.

### Example 1

### Optimization of Gene Signatures

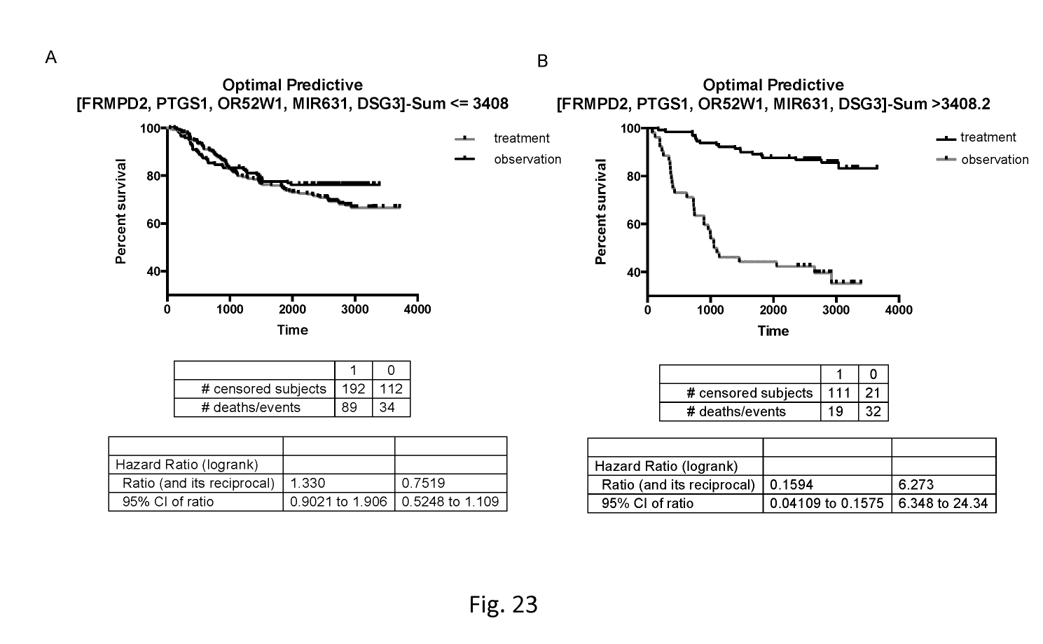

To determine biologically relevant optimal gene signatures, the inventors have selected for gene signatures that share a common known feature or attribute such as sharing a common DNA promoter motif indicating a shared transcription factor responsible for differential expression of the gene signature.

[FRMPD2, PTGS1, OR52W1, MIR631, DSG3] shown in (FIG. 23) ranked #1 as a predictive gene signatures (HR=0.159 95% CI=0.04-0.16) for the sum of the mRNA expression resulting in a high expression group as compared to the cohort mean benefiting from treatment with trastuzumab, a significant improvement over the [C8A] gene signature with a HR=0.246

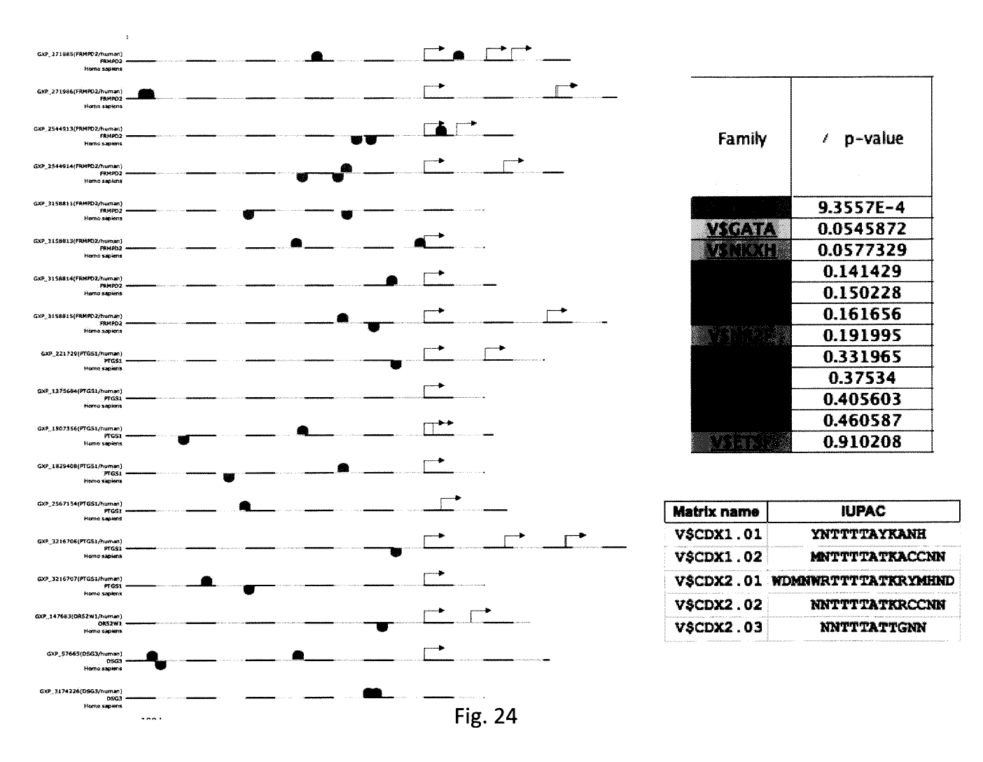

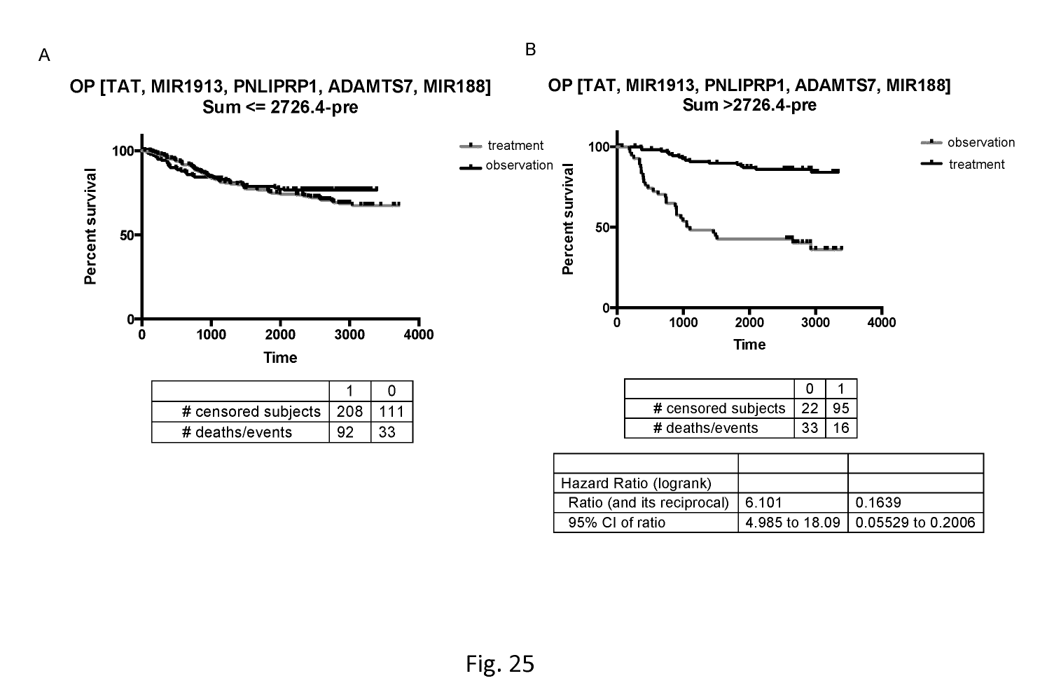

Promoter analysis of [FRMPD2, PTGS1, OR52S1, DSG3] shown in (FIG. 24) indicates a shared promoter family V$CDXF with a p-value of 9.3E-4. In total, 4 of the 5 genes in the gene signature share a common promoter element. MIR631 is not included in the promoter analysis as it is a miRNA and is not annotated in the software. The V$CDXF promoter motifs are docking sites for CDX1, CDX2 and CDX4 for example. The unique combination of the gene signature where each gene shares a common promoter motif indicates the biological relevance and significance of the gene signature.

[TAT, MIR1913, PNLIPRP1, ADAMTS7, MIR188] shown in (FIG. 25) is the second best optimized predictive signature with no genes shared with [FRMPD2, PTGS1, OR52S1, DSG3] (HR=0.164 95% CI=0.06-0.20) where the patients with high expression of the sum of the mRNA for each gene in the signature as compared to the cohort mean benefits from treatment with trastuzumab.

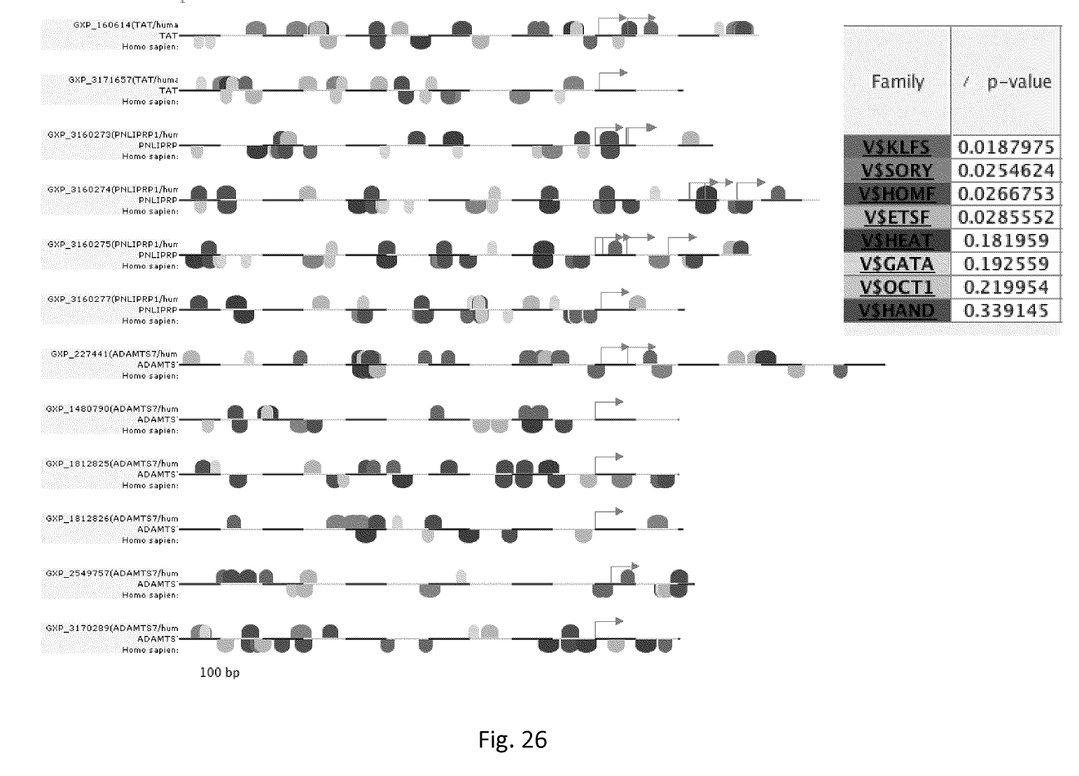

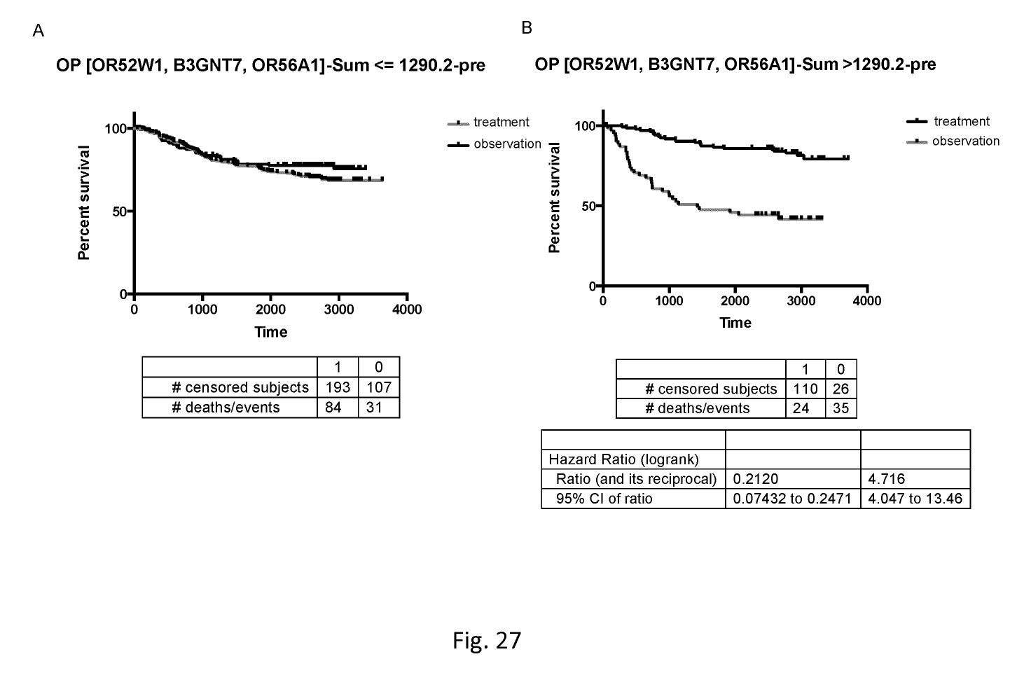

The three genes [TAT, PNLIPRP1, ADAMTS7] have promoter patterns in common V$KLFS (p=0.019), V$SORY (p=0.026), V$HOMF (p=0.027), V$ETSF (p=0.029) shown in (FIG. 26). The unique combination of the gene signature where each gene shares a common set of promoter motifs indicates the biological relevance and significance of the gene signature.

[OR52W1, B3GNT7, OR56A1] shown in (FIG. 27) has no genes in common with [FRMPD2, PTGS1, OR52S1, DSG3] or [TAT, MIR1913, PNLIPRP1, ADAMTS7, MIR188] with a resulting predictive (HR=0.164 95% CI=0.06-0.20) where the patients with high expression of the sum of the mRNA for each gene in the signature as compared to the cohort mean benefits from treatment with trastuzumab.

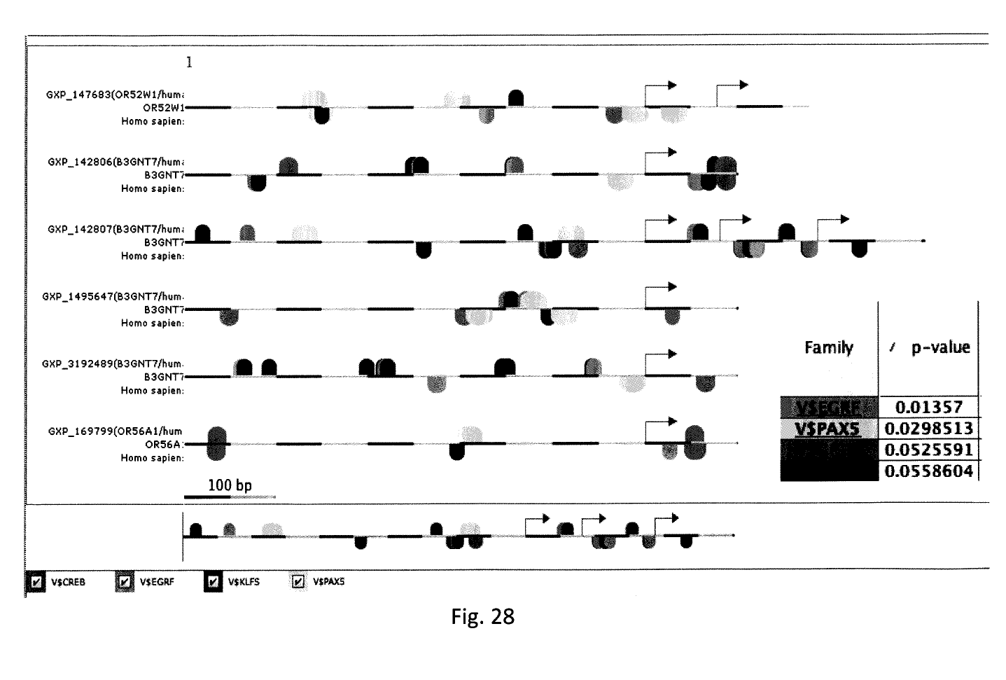

The three genes [OR52W1, B3GNT7, OR56A1] have promoter patterns in common V$EGRF (p=0.014), V$PAX5 (p=0.03), V$CREB (p=0.053) and V$KLFS (p=0.056) shown in (FIG. 28). The unique combination of the gene signature where each gene shares a common set of promoter motifs indicates the biological relevance and significance of the gene signature.

The exploratory analysis of predictive single gene biomarkers allows for the determination of optimal gene signatures with improved hazard ratios over any single gene. Numerous combinations of optimal predictive gene signatures exist as a result of the exploratory analysis where unique combinations of genes that share a common biological attribute such as promoter motif, molecular GO annotation, canonical pathway, chromosomal location etc. indicates a biologically relevant gene signature.

Numerous methods can be used to determine biomarkers or gene signatures of interest in a particular cohort that do not validate when tested in other cohorts.

By requiring gene signatures to have a common biological attribute such as a DNA promoter motif the biological relevance of the gene signature is indicated and reduces the likelihood that the gene signature results from over fitting or occurred by chance.

While preferred embodiments have been described above and illustrated in the accompanying drawings, it will be evident to those skilled in the art that modifications may be made without departing from this disclosure. Such modifications are considered as possible variants comprised in the scope of the disclosure.

1. A method for prognosis of breast cancer outcome and/or predicting the benefit of treatment with an anti-ErbB antibody, comprising the step of:
(a) providing information on breast cancer treatment based on a comparison of mRNA expression level of a gene signature consisting of at least one biomarker chosen from C8A, OR56A1, PRR20C, B3GNT7, MIR609, ADAMTS7, MIR188, DSG3, FLJ45139, FRMPD2, MIR1913, MIR631, PNLIPRP1, TAT, ZNF528, OR52W1, LOC100131187, PTGS1, GFI1B, and TLX2 in a test sample from a subject, to a reference expression level of said biomarker,
wherein said biomarker expression level is quantified using a biological assay;
wherein over expression of the identified biomarker in the test sample indicates a poor breast cancer outcome and a beneficial treatment with said anti-ErbB antibody, and
wherein minimal expression, or under expression of the biomarker in the test sample indicates positive outcome and a lack of benefit of treatment with said anti-ErbB antibody.

2. The method of claim 1, further comprising step (b) when said gene signature comprises at least two biomarkers:
(b) optimizing said gene signature by identifying a common biological attribute between said at least two biomarker, to provide an optimized gene signature,
wherein over expression in the test sample of the at least two biomarkers identified in said optimized gene signature indicates a poor breast cancer outcome and a beneficial treatment with ErbB antibodies, and
wherein minimal expression, or under expression in the test sample of the at least two biomarkers identified in said optimized gene signature indicates positive outcome and a lack of benefit of treatment with ErbB antibodies.

3. The method of claim 1, wherein said biomarker is one of C8A, OR56A1, PRR20C or a combination of C8A.

4. The method of claim 1, wherein said biomarker is C8A.

5. A method for treatment of cancer in an individual comprising:
a) determining, in a tumor sample from said patient, a value for at least one biomarker chosen from C8A, OR56A1, PRR20C, B3GNT7, MIR609, ADAMTS7, MIR188, DSG3, FLJ45139, FRMPD2, MIR1913, MIR631, PNLIPRP1, TAT, ZNF528, OR52W1, LOC100131187, PTGS1, GFI1B, and TLX2;
b) comparing the value obtained in step a) for said biological marker with a predetermined reference value for the same biomarker; and
c) administering a therapeutic agent to said individual if the value of said biomarker is above/below the predetermined reference value.

6. A method of identifying a cancer patient suitable for treatment with a therapeutic agent, comprising:
a) determining, in a tumor sample from said patient, a value for at least one biomarker chosen from C8A, OR56A1, PRR20C, B3GNT7, MIR609, ADAMTS7, MIR188, DSG3, FLJ45139, FRMPD2, MIR1913, MIR631, PNLIPRP1, TAT, ZNF528, OR52W1, LOC100131187, PTGS1, GFI1B, and TLX2;
b) comparing the value obtained in step a) for said biological marker with a predetermined reference value for the same biomarker; and
c) identifying the cancer patient as suitable for treatment with the therapeutic agent if the value of said biomarker is above/below the predetermined reference value.

7. A method for the prognosis of cancer, comprising:
a) determining, in a tumor sample from said patient, a value for at least one biomarker chosen from C8A, OR56A1, PRR20C, B3GNT7, MIR609, ADAMTS7, MIR188, DSG3, FLJ45139, FRMPD2, MIR1913, MIR631, PNLIPRP1, TAT, ZNF528, OR52W1, LOC100131187, PTGS1, GFI1B, and TLX2; and
b) comparing the value obtained in step a) for said biomarker with a predetermined reference value for the same biological marker, which predetermined reference value is correlated with a specific prognosis of cancer;
wherein when the value of said biomarker is above the predetermined reference value, prognosis of said cancer outcome is a bad prognosis; and
wherein when the value of said biomarker is below the predetermined reference value, prognosis of said cancer outcome is a good prognosis.

8. The method of any of claims 14 to 6, wherein the method is an in vitro method.

9. The method of any one of claims 5 to 8, further comprising step (b) when said gene signature comprises at least two biomarkers:
(b) optimizing said gene signature by identifying a common biological attribute between said at least two biomarker, to provide an optimized gene signature,
wherein over expression in the test sample of the at least two biomarkers identified in said optimized gene signature indicates a poor breast cancer outcome and a beneficial treatment with ErbB antibodies, and
wherein minimal expression, or under expression in the test sample of the at least two biomarkers identified in said optimized gene signature indicates positive outcome and a lack of benefit of treatment with ErbB antibodies.

10. The method of any one of claims 5 to 8, wherein said biomarker is one of C8A, OR56A1, or PRR20C, or a combination of C8A, OR56A1 and PRR20C.

11. The method of any one of claims 5 to 8, wherein said biomarker is C8A.

12. The method of any one of claims 1 to 9, wherein said biomarker is a combination of FRMPD2, PTGS1, OR52W1, MIR631, and DSG3, or a combination of TAT, MIR1913, PNLIPRP1, ADAMTS7, and MIR188, or a combination of OR52W1, B3GNT7, and OR56A1.

13. The method of any one of claims 5 to 12, wherein said cancer is breast cancer.

14. The method of any one of claim 5 or 6, wherein when said value of said biomarker is above the predetermined reference value, said therapeutic agent is an anti-ErbB antibody.

15. The method of any one of claim 5 or 6, wherein when said value of said biomarker is below the predetermined reference value, said therapeutic agent is different than an anti-ErbB antibody.

16. The method of claim 14, further comprising treatment with an additional therapeutic agent.

17. The method of any one of claims 15 and 16, wherein said therapeutic agent different than an anti-ErbB antibody or said additional therapeutic agent is at least one of doxorubicin (Adriamycin®), Liposomal doxorubicin (Doxil®), epirubicin (Ellence®), taxanes such as paclitaxel (Taxol®), docetaxel (Taxotere®), Albumin-bound paclitaxel (nab-paclitaxel or Abraxane®), fluorouracil (5-FU), cyclophosphamide (Cytoxan®), platinum agents such as cisplatin and carboplatin, Vinorelbine (Navelbine®), Capecitabine (Xeloda®), Gemcitabine (Gemzar®), Mitoxantrone; Ixabepilone (Ixempra®), Eribulin (Halaven®).

18. The method of any one of claims 5 to 17, wherein said value for a biological marker is an expression level.

19. The method of any one of claims 5 to 17, wherein said determining is with a biological assay.

20. The method of claim 19, wherein said biological assay is at least one of PCR based methods, hybridization based methods, sequencing methods, protein detection methods, or combinations thereof.

21. The method of claim 20, wherein the PCR based methods comprise reverse transcriptase polymerase chain reaction (RT-PCR), and quantitative reverse transcriptase polymerase chain reaction (QRT-PCR).

22. The method of claim 20, wherein the hybridization methods comprise nuclease protection assay, Northern blot analysis, in situ hybridization, and microarray based analysis.

23. The method of claim 20, wherein the sequencing methods comprises next generation sequencing (NGS) technologies.

24. The method of claim 20, wherein the protein detecting methods comprises a Western blot analysis, an enzyme-linked immunosorbent assay (ELISA), immunohistochemistry analysis, an immunoprecipitation followed by an SDS-PAGE analysis, a proteomic analysis.

25. The method of claim 18, wherein said expression level is one of an mRNA expression level, a protein expression level, or combinations thereof.

26. The method of any one of claims 5 to 17, wherein said value for a biological marker is a gene copy number.

27. The method of claim 7, wherein when said value of said biomarker is above the predetermined reference value, said bad prognosis is indicative of a beneficial treatment with an anti-ErbB antibody.

28. The method of claim 7, wherein when said value of said biomarker is below the predetermined reference value said good prognosis is indicative of a non-beneficial treatment with an anti-ErbB antibody.

29. The method of any one of claims 1 and 14, wherein said anti-ErbB antibody is one or more than one anti-ErbB antibody.

30. The method of claim 29, wherein said one or more anti-ErbB antibody is Pertuzumab, Trastuzumab, T-DM1 or combinations thereof.

31. The method of any one of claims 1 to 30, wherein said reference value for the at least one biomarker or said reference expression level of said biomarker is determined by a method comprising the steps:
a) providing at least one collection of tumor samples selected from the group consisting of:
i) a collection of tumor samples from cancer patients with a known cancer classification, having undergone anti-cancer treatment, and subsequently having no cancer relapse or no cancer recurrence after the anti-cancer treatment;
ii) a collection of tumor samples from cancer patients with a known cancer classification, having undergone anti-cancer treatment, and subsequently having cancer relapses or recurrences after the anti-cancer treatment.
b) quantifying, for each sample comprised in a collection of tumor samples provided at step a), said at least one biomarker, whereby a collection of quantification values for said at least one biomarker and for the said collection of tumor samples is obtained;
c) calculating, from the said collection of quantification values obtained at the end of step b), the mean quantification value for said at least one biomarker, whereby a predetermined reference value for said at least one biomarker that is correlated with a specific cancer prognosis is obtained.

32. The method of any one of claims 1 to 30, wherein said reference value for the at least one biomarker or said reference expression level of said biomarker is a reference cut-off value determined by a method comprising the steps:
a) selecting at least one biomarker for which a reference value is to be determined;
b) providing a collection of tumor tissue samples from cancer patients;
c) providing, for each tumor sample provided at step b), information relating to the actual clinical outcome for the corresponding cancer patient;
d) providing a serial of arbitrary quantification values for said at least one biomarker selected at step a);
e) quantifying said at least one biomarker in each tumor tissue sample contained in the collection provided at step b);
f) classifying said tumor samples in two groups for one specific arbitrary quantification value provided at step c), respectively:
(i) a first group comprising tumor samples that exhibit a quantification value for said at least one biomarker that is lower than said arbitrary quantification value contained in said serial of quantification values;
(ii) a second group comprising tumor samples that exhibit a quantification value for said at least one biomarker that is higher than said arbitrary quantification value contained in said serial of quantification values;
whereby two groups of tumor samples are obtained for said specific quantification value, wherein the tumors samples of each group are separately enumerated;
g) calculating the statistical significance between (i) the quantification value for said at least one biomarker obtained at step e) and (ii) the actual clinical outcome of the patients from which tumor samples contained in the first and second groups defined at step f) derive;
h) reiterating steps f) and g) until every arbitrary quantification value provided at step d) is tested;
i) setting said reference cut off value as consisting of the arbitrary quantification value for which the highest statistical significance (most significant) has been calculated at step g).

33. The method of claim 32, wherein in step c), said information relating to the actual clinical outcome of the patients is selected from the group consisting of the duration of the disease-free survival (DFS) and the overall survival (OS), and a combination thereof.

34. The method of claim 32, wherein the reference cut-off value is a median quantification value for said at least one biomarker that discriminates between bad cancer prognosis and good cancer prognosis.

35. The method of any one of claims 1 to 30, wherein when said at least one biomarker is an expression level of a gene, said reference value of the gene expression value or said reference expression level of said biomarker correlates with bad cancer prognosis.

36. A kit for the prognosis of breast cancer outcome and/or predicting the benefit of treatment with an anti-ErbB antibody, comprising
(a) an agent that specifically detects a biomarker, wherein said biomarker is at least one biomarker chosen from C8A, OR56A1, PRR20C, B3GNT7, MIR609, ADAMTS7, MIR188, DSG3, FLJ45139, FRMPD2, MIR1913, MIR631, PNLIPRP1, TAT, ZNF528, OR52W1, LOC100131187, PTGS1, GFI1B, and TLX2; and
(b) instructions to use said kit.

37. A kit for identifying a cancer patient suitable for treatment with a therapeutic agent comprising:
(a) an agent that specifically detects a biomarker, wherein said biomarker is at least one biomarker chosen from C8A, OR56A1, PRR20C, B3GNT7, MIR609, ADAMTS7, MIR188, DSG3, FLJ45139, FRMPD2, MIR1913, MIR631, PNLIPRP1, TAT, ZNF528, OR52W1, LOC100131187, PTGS1, GFI1B, and TLX2; and
(b) instructions to use said kit.

38. A kit for the prognosis of cancer comprising:
(a) an agent that specifically detects a biomarker, wherein said biomarker is at least one biomarker chosen from C8A, OR56A1, PRR20C, B3GNT7, MIR609, ADAMTS7, MIR188, DSG3, FLJ45139, FRMPD2, MIR1913, MIR631, PNLIPRP1, TAT, ZNF528, OR52W1, LOC100131187, PTGS1, GFI1B, and TLX2; and
(b) instructions to use said kit.

39. The kit of any one of claims 36 to 38, wherein said an agent that specifically detects a biomarker is a nucleic acid probe, a protein binding agent, or a combination thereof.

40. The kit of claim 39, wherein said nucleic acid probe is a pair of nucleic acid probe.

41. The kit of claim 39, wherein said protein binding agent is an antibody, an antibody fragment, an aptamer, or a combination thereof.

42. The kit of any one of claims 36 to 41, wherein said biomarker is C8A.
